(19) United States
(12) Patent Application Publication
(10) Pub. No.: US 2009/0308398 A1
(43) Pub. Date: Dec. 17, 2009

(54) ADJUSTABLE RESISTANCE NASAL DEVICES

(76) Inventors: Arthur Ferdinand, San Jose, CA (US); Danny Yu-Youh Lai, San Jose, CA (US); Michael Pou Wong, San Francisco, CA (US); Elliot Sather, San Francisco, CA (US); Michael L. Favet, San Jose, CA (US); Rajiv Doshi, Los Altos, CA (US)

Correspondence Address: SHAY GLENN LLP, 2755 CAMPUS DRIVE, SUITE 210, SAN MATEO, CA, 94403, US

(21) Appl. No.: 12/485,750

(22) Filed: Jun. 16, 2009

Related U.S. Application Data
(60) Provisional application No. 61/061,918, filed on Jun. 16, 2008.

(51) Int. Cl. A61M 16/20

(52) U.S. Cl. 128/207.18

(57) ABSTRACT

Described herein are adjustable-resistance respiratory devices, and particularly nasal devices that have an adjustable expiratory resistance while providing a greater resistance to exhalation than to inhalation. The resistance to exhalation may be manually adjustable by a user or remotely adjustable by a third party. For example, described herein are nasal devices having a greater resistance to exhalation than inhalation that includes one or more resistance-modifying members for modifying the resistance of a nasal device. A resistance modifying member may include a cover, a shutter or an adjustable valve for opening/closing a leak pathway through the nasal device. An adjustable-resistance nasal respiratory device may include a control or controls for adjusting the resistance to exhalation. Methods of adjusting the resistance of a nasal device, and systems including nasal devices allowing the resistance to be optimized and/or adjusted are also described.

### CROSS-REFERENCE TO RELATED APPLICATIONS

This application claims priority to U.S. Provisional Patent Application Ser. No. 61/061,918, titled “Adjustable Resistance Nasal Devices,” filed on Jun. 16, 2008. This application is herein incorporated by reference in its entirety.

This application may be related to pending U.S. patent application Ser. No. 11/298,339, titled “Respiratory Devices”, filed Dec. 8, 2005, which claims priority to U.S. Provisional Patent Application Ser. No. 60/634,715, filed Dec. 8, 2004. This application may also be related to pending U.S. patent application Ser. No. 11/805,496, titled “Nasal Respiratory Devices”, filed May 22, 2007, which claims priority to U.S. Provisional Patent Application Ser. No. 60/808,034, filed May 23, 2006.

### BACKGROUND OF THE INVENTION

Nasal respiratory devices may be worn to treat many medical conditions, such as sleep disordered breathing (including snoring, sleep apnea, etc.), Cheyne Stokes breathing, UARS, COPD, hypertension, asthma, GERD, heart failure, and other respiratory and sleep conditions. Devices that provide a greater resistance to exhalation than to inhalation may be particularly useful, and may be worn by a subject when the subject is either awake or asleep. Indeed, subjects may apply a nasal device before falling to sleep, so that the device may provide therapeutic benefits during sleep. However, optimal levels of resistance to exhalation (and/or inspiration) may be different for individual users, or for the same user over the course of treatment for a particular user, and even over the course of a single treatment session. In some instances, the optimal resistance may be determined by adjusting the resistance while the subject is sleeping with the device (e.g., without waking the subject).

Examples of nasal respiratory devices have been well-described in the following US patents and patent applications, each of which is incorporated herein in its entirety: U.S. patent application Ser. No. 11/298,640, titled “Nasal Respiratory Devices”) filed Dec. 8, 2005; U.S. patent application Ser. No. 11/298,339, titled “Respiratory Devices”, filed Dec. 8, 2005; U.S. patent application Ser. No. 11/298,362, titled “Methods of Treating Respiratory Disorders”, filed Dec. 8, 2005; U.S. patent application Ser. No. 11/805,496, titled “Nasal Respiratory Devices”, filed May 22, 2007; U.S. Pat. No. 7,506,649, titled “Nasal Devices”, filed Jun. 7, 2007; U.S. patent application Ser. No. 11/759,916, titled “Layered Nasal Devices” filed Jun. 7, 2007; U.S. patent application Ser. No. 11/811,401, titled “Nasal Respiratory Devices for Positive End-Expiratory Pressure”, filed Jun. 7, 2007; U.S. patent application Ser. No. 11/941,915, titled “Adjustable Nasal Devices”, filed Nov. 19, 2007; and U.S. patent application Ser. No. 11/941,913, titled “Nasal Device Applicators”, filed Nov. 16, 2007.

Such nasal respiratory devices may passively induce positive end-expiratory pressure (“PEEP”) or expiratory positive airway pressure (“EPAP”), and are adapted to be removably secured in communication with a nasal cavity. These devices act passively because they do not actively apply positive airflow, but instead regulate the subject's normal breathing, typically using one or more valves to inhibit exhalation more than inspiration. These nasal respiratory devices are adapted to be removably secured in communication with a nasal cavity, and may include a passageway (which may just be an opening) through the device, a valve (or airflow resistor) in communication with the passageway, and a holdfast. The holdfast is configured to removably secure the respiratory device at least partly within (and/or at least partly over and/or at least partly around) the nasal cavity. The airflow resistor (which may be a valve) is typically configured to provide greater resistance during exhalation than during inhalation.

Examples of these devices are shown in FIGS. 1A-2B, and are briefly described below. Exemplary nasal devices may include an airflow resistor (e.g., a flap valve or multiple flap valves) providing a greater resistance to exhalation than to inhalation, a holdfast to secure the nasal device in communication with the subject's nose, and optionally a rim body forming a passageway in which the airflow resistor is positioned, and an aligner for aligning the device with respect to one or more of the subject's nostrils. In general, these nasal respiratory devices may be configured so that the airflow resistor provides a resistance to exhalation that is between about 10 cm H₂O*sec/L and about 250 cm H₂O*sec/L (e.g., 0.01 and about 0.25 cm H₂O/(ml/sec)) when measured at 100 ml/sec rate of airflow, and a resistance to inhalation that is between about 0.1 cm H₂O*sec/L and about 20 cm H₂O*sec/L (e.g., 0.0001 and about 0.02 cm H₂O/(ml/sec)) when measured at a rate of airflow of 100 ml/sec. For example, FIGS. 1A and 1B show front and back perspective views (respectively) of one variation of an adhesive nasal device.

For example, the nasal device shown in FIGS. 1A and 1B are two single-nostril devices that have been joined to form a single device. In similar variations the two single-nostril devices are not joined by this bridge region 112, but are kept separate, and may be applied separately to each nostril. In some variations, both nostrils may be covered by a single airflow resistor or region including one or more airflow resistors, as described in greater detail below. The front view of the nasal device shown in FIG. 1A illustrates the outward-facing side of this variation of a nasal device, when it is worn by a subject.

FIGS. 1A-2B show examples of nasal devices that may be adapted to include one or more adjustable resistance features as described herein. When operating these nasal devices, some users may benefit from adjusting the resistance to exhalation through the nasal device. Other examples of nasal devices including airflow resistors are shown in FIGS. 3A-3G. Each of these devices is configured so that it inhibits exhalation through the nose (one or both nostrils) more than it inhibits inhalation. In any of these devices, it would be useful to provide devices for which the resistance to exhalation and/or the resistance to inspiration may be adjusted or adjustable. Described and illustrated below are nasal respiratory devices that may allow adjustable expiratory and/or inspiratory resistance.

Adjustable-resistance nasal devices may be particularly beneficial to determine (e.g., by a sleep study) the appropriate resistance(s) to exhalation (and/or inhalation) for a respiratory device that inhibits exhalation more than inhalation. Although the majority of devices described herein refer to nasal devices, the invention and principles described herein may be adapted for use with respiratory devices that including oral masks, and combined oral/nasal masks, including PAP valve masks. Adjustable-resistance nasal devices may also be used or by a subject within a single treatment or between treatment days. For example, a subject may increase the resistance to exhalation manually (himself or herself) as the subject acclimates to the device.

The devices and methods described herein address the needs and concerns referred to above.

### SUMMARY OF THE INVENTION

Described herein are nasal respiratory devices including devices configured to have an adjustable resistance. In general, nasal respiratory devices and nasal devices having an adjustable resistance may allow adjustment of either (or both) the resistance to inhalation and the resistance to exhalation. In particular, described herein are adjustable nasal devices configured to allow adjustment of the resistance to exhalation (“expiratory resistance”). The adjustable nasal respiratory devices described herein may be referred to as “adjustable-resistance nasal devices” or simply “adjustable nasal devices” although they may include additional features in addition to the resistance-adjustment features. The resistance of these adjustable resistance nasal devices may be manually or automatically adjusted. In some variations, the resistance is remotely adjustable (e.g., by a third party), and may be adjusted while the subject is sleeping. The adjustable devices may have their resistance adjusted by altering and controlling the size (e.g., cross-sectional area) of one or more leak pathways through the devices described herein. In particular, the adjustment of the resistance (e.g., the resistance to exhalation) may be adjusted by increasing or decreasing the size or number leak pathways that are independent of the airflow resistor(s) in the device.

In some variations, the adjustable resistance nasal devices described herein are nasal devices having flap valve airflow resistors. As described in greater detail below, a flap valve is generally a flat structure having two opposing faces and a minimal thickness that substantially opens during inhalation and closes during exhalation. Although the airflow resistors described herein are primarily flap-valve type airflow resistors, any appropriate airflow resistor may be used, including non-flap valve airflow resistors.

An adjustable respiratory device as described herein may be continuously adjustable between a range of resistances. For example, the adjustable devices may allow adjustment of expiratory resistance within a range that is between about 0.01 and about 0.25 cm H₂O/(ml/sec) when measured at 100 ml/sec. In some variations the resistance to inhalation (“inspiratory resistance”) may be separately adjustable. For example, the resistance to inhalation is less than the resistance to exhalation, and may be adjustable within a range of between about 0.0001 and about 0.02 cm H₂O/(ml/sec) when measured at 100 ml/sec. In general, however, the adjustable-resistance nasal devices described herein, the adjustability of the resistance typically refers to adjusting the resistance to exhalation.

In some variations, an adjustable resistance respiratory device is adjustable to predetermined settings or steps. For example, the expiratory resistance of an adjustable resistance nasal device may be adjustable in increments of 0.005 cm H₂O/(ml/sec) when measured at 100 ml/sec. In some variations, the device may be adjustable between, two, three, four, five, six, or more expiratory resistance levels.

The adjustable respiratory devices described herein typically include one or more leak pathways that are configured to remain open during both inhalation and exhalation. During operation of the nasal devices described herein, the airflow resistor (e.g., flap valve(s)) are typically closed during exhalation, increasing the resistance within the target range, and the flap valve(s) of the airflow resistor are typically at least partly open during inhalation. In general, the resistance to exhalation may be adjusted by controlling either or both the closing of the airflow resistor and/or the leak pathways. Adjustment of the expiratory resistance by controlling the leak pathways that are independent of the airflow resistor may be particularly useful, since changing the closing state of the airflow resistor may make the expiratory resistance difficult to control. In addition, adjustment of the airflow resistor may be used to adjust the resistance to inhalation, since modification of the airflow resistor may modify the opening of the airflow resistor during inhalation.

In general, adjustable-resistance nasal respiratory devices have a resistance to exhalation that is greater than the resistance to inspiration. In some variations, the resistance to inspiration is relatively constant (i.e., pre-set), while the resistance to exhalation may be adjusted. In other variations, both the resistance to exhalation and the resistance to inspiration are adjustable. In still other variations, the resistance to inspiration is adjustable while the resistance to exhalation is pre-set. Although the majority of examples provided herein refer only to devices and methods for adjusting the expiratory resistance, many of the same principles and techniques described may be applied to allow adjusting of the inspiratory resistance.

As used herein, the term “adjusting” or “adjustable” typically refers to modifying or changing the resistance of a nasal respiratory device. An adjustment may be made dynamically (e.g., while the device is being worn), or it may be made prior to applying the device to a subject or patient. As mentioned above, an adjustable device may be continuously adjustable, so that the resistance (e.g., to exhalation) may be transitioned continuously over a range, or it may be discretely adjustable, so that the resistance may be transitioned in steps. The adjustable devices may be user- or subject-adjustable, and may include one or more controls (e.g., knobs, buttons, dials, wheels, etc.). In some variations the adjustable devices are remotely adjustable. Adjustable devices may be adjusted by the application or removal of a modifying member or component (e.g., a snap-on resistance modifying member, an adhesive resistance modifying member, etc.). Any of the resistance modifying members that attach to the nasal device may also be attached to a nasal cannula or sensor (e.g., thermister, airflow sensor, pressure sensor, etc.) or may be adapted for use with such a sensor or sensing element.

In some variations, the resistance to exhalation may be modified by controlling the number, size and/or shape of a leak pathway (or pathways) through the device. As used herein, the term “leak pathway” may refer to an opening or channel through the device that is open when the airflow resistor is closed. A leak pathway may be independent (and separate from) the airflow resistor. In some variations a leak pathway is formed around the airflow resistor (e.g., flap or membrane valve).

In general, the nasal devices having an adjustable resistance typically include an airflow resistor (which may comprise, for example, a flap valve) that is configured to inhibit exhalation more than inhalation, and a holdfast configured to secure the nasal device in communication with one or more of the subject's nostrils. The nasal devices may also include one or more leak pathways or openings that are typically open during both exhalation and inhalation. As mentioned, an adjustable-resistance nasal device may include any appropriate airflow resistor, including (but not limited to) flap or diaphragm valves, ball valves, duckbill valves, hinge-less valves, balloon valves, stepper valves, slit valves, PEEP valves, threshold valves, etc., or the like. In addition, any of the adjustable-resistance nasal devices described herein may include any appropriate holdfast for securing the device in communication with the subject's nose. For example, any of these devices may be adhesive nasal devices, which include one or more adhesive holdfasts or may be mask devices that fit over the nose and/or the mouth.

The adjustable resistance nasal devices described herein may be adjustable within any appropriate treatment range, including those described above. For example, an adjustable resistance nasal device may be adjustable so that the resistance to exhalation can be set to between about 1 and about 250 cm H₂O/(l/sec). In some variations, the resistance to exhalation can be set between about 5 and about 250 cm H₂O/(l/sec). The nasal devices described herein may have a very low resistance to inspiration. For example, the resistance to inspiration may be between about 0.01 and about 5 cm H₂O/(l/sec) (and in adjustable resistance nasal devices configured to allow adjustment of the inhalational resistance, the resistance to inhalation may be varied within this range). As mentioned below, the adjustment may be continuous (over a range or resistances) or it may be discrete (in steps), or some combination of the two. The adjustment may be linear or non-linear.

In some variations, an adjustable resistance nasal device includes a leak pathway that can be plugged or covered. The leak pathway cover may be integrated as part of the nasal device, or it may be a separate component or structure that can be applied to the nasal device to occlude or partially occlude the leak pathway and thereby increase the resistance to exhalation (or be removed from the nasal device to decrease resistance to exhalation). For example, the device may include a snap-on or adhesive cover for covering one or more leak pathways. In some variations, the cover is adjustable so it only partially occludes the leak pathway. An example of an adhesive plug or cover may be a piece of tape or adhesive strip that can be used to cover the leak pathway. In some variations the cover or plug is attached (e.g., by a tether, hinge, etc.) to the nasal device. In some variations the plug is integral to the device and may be pushed (e.g., by a finger) to activate and increase the resistance (and pulled to decrease the resistance).

In general, in any of the variations described herein, the resistance (e.g., to exhalation) may be modulated by controlling the amount of a leak pathway occluded/opened, or the number of leak pathways opened or occluded. If a device has multiple leak pathways, the resistance may be stepped up by blocking increasing numbers of the leak pathways. In any of these variations, the nasal devices may include adjustable controls that are calibrated as to the resistance (e.g., expiratory resistance). For example, a snap-fit cover to increase resistance may be labeled or otherwise marked to indicate the resistance (or range of resistances) that the nasal device will have after applying the cover. This general principle may be applied to any of the nasal devices or components used to modulate the resistance described herein. For example, a control for continuously or discretely adjusting the resistance may include markings or settings to indicate the resistance.

In some variations, an adjustable resistance nasal device may include a leak pathway that is directly adjustable by changing the size or shape of the leak pathway opening. For example the leak pathway may be adapted to constrict (e.g., by including an inflatable or swellable material). In some variations the leak pathway may include a shutter or cover that may be used to close it off, or partially close it off. For example, the leak pathway may include a louver-type cover or shutter that can be moved to partially or completely occlude the opening of one or more leak pathways. In some variations the leak pathway includes an iris (e.g., a dilating iris) that can be used to cover or open the leak pathway. In any of these embodiments, the device may include one more handles/controls for manually operating the closing and/or opening of the leak pathway or may include electronic means of closing and/or opening the leak pathway, especially from a remote location (for example in the control room of a sleep laboratory).

Also described herein are nasal devices in which the position of all or a part of the airflow resistor may be adjusted to modify the resistance. For example, the position of the airflow resistor may be modified relative to a passageway through the device. In some variations the registration of the airflow resistor relative to the passageway may be changed, to increase/decrease the size of a leak pathway at least partially around the airflow resistor. For example, the airflow resistor may include a flap valve that can be rotated slightly relative to the passageway. In some variations the airflow resistor is a flap valve that can be shifted with respect to the flap valve limiter (e.g., supports or struts) across a passageway, so that the flap valve can be seated in different positions that allow more or less air to pass through the passageway (leak) when the valve is closed during exhalation. In some variations the proximal/distal position of the airflow resistor may be changed. For example, the airflow resistor may be moved proximally or distally along the length of a tapered passageway. As the device moves in the direction of the narrowing of the tapered passageway (e.g., proximally) less air may pass around the device, thereby increasing the leak size and the thus the resistance to exhalation. In some variations movement of the airflow resistor (or a portion of the airflow resistor) may be controlled by a control such as a knob. For example, a worm-screw type control may be used to move the airflow resistor proximally or distally in some variations. In some variations, the nasal device includes one or more leak pathways as part of the nasal device. For example, the airflow resistor may include a flap valve having one or more holes (leak pathways). The expiratory resistance may be adjustable by rotating the flap valve so that the holes on the flap valve are partially occluded (or un-occluded) when the flap valve is closed during exhalation. For example, the holes may be aligned with a portion of the flap valve limiter (e.g., struts, mesh, etc.) that blocks the holes closed when the valve is closed.

Also described herein are adjustable resistance nasal devices in which the operation of the airflow resistor is modified. For example, device may be adapted so that the airflow resistor (e.g., flap valve) is prevented to a controllable degree from closing completely during exhalation. In some variations the device includes one or more adjustable members that prevent the edge of the valve from fully closing during exhalation by propping the valve open. In some variations the device includes an adjustable member that raises or lowers the hinge or pivot portion of the valve so that the valve cannot seat closed (completely) during exhalation.

Also described herein are adjustable resistance nasal devices in which the length of the leak pathway is adjustable (e.g., can be increased and/or decreased). For example, the length of the leak pathway can be decreased by removing a section of the leak pathway to decrease the resistance during exhalation. In some variations the leak pathway is a telescoping channel that can be elongated or shortened.

Methods of adjusting the resistance, and particularly the expiratory resistance, are also described. In general, any of the devices described herein, alone or in combination, can be used to adjust or control (e.g., increase or decrease) the resistance to exhalation through the devices. These devices may be used to optimize treatment of disorders such as sleeping disorders, as described briefly above.

Also described herein are systems for adjusting the resistance of a nasal device. In particular, a system may include any of the nasal devices described herein and any cover for altering the expiratory resistance (e.g., a snap-on cover or plug, etc.).

A system for optimizing the resistance to exhalation may include a plurality of nasal devices having progressively increasing or decreasing resistances to exhalation. Such a system may be used to determine a patient-specific resistance for exhalation. In use, a subject may sequentially wear nasal devices having different expiratory resistance to determine or optimize comfort and/or efficacy of treatment.

In particular, described herein are systems or kits having a plurality of nasal devices each with increasing resistances to exhalation (and/or inspiration). The kit may include instruction to the user indicating the order in which each of the nasal devices is to be worn for a particular number of nights. Such a systems or kits may be referred to as “ramp systems”, “ramp kits,” “acclimation systems ” or “acclimation kits.” For example, a system may include a first device or set of devices having a very low resistance to exhalation (e.g., less than 20 cm H₂O/(L/sec)) or range of expiratory resistances, a second device or set of devices having a resistance to exhalation (or range of expiratory resistance) that is slightly higher (e.g., approximately 30 cm H₂O/(L/sec)), a third device or set of devices having a slightly higher yet resistance to exhalation (e.g., approximately 40 cm H₂O/(L/sec)) or range of expiratory resistance, a fourth device or set of devices having a slightly higher resistance to exhalation than the third device or set of devices (e.g., approximately 50 cm H2O/(L/sec)) or range of expiratory resistance, a fifth device or set of devices having a slightly higher resistance to exhalation than the fourth device or set of devices (e.g., approximately 60 cm H2O/(L/sec)), or range of expiratory resistance, etc. so that the resistance of the next device or set of devices in the series is slightly higher than the previous device or set of devices. These first, second, third, etc. devices or set of devices are marked to indicate their order in the sequence (or are packaged to indicate their order in the sequence). The first device or set of devices in the sequence may be a ‘sham’ device, which does not include a significant resistance to exhalation compared to inhalation. The instructions may indicate the number of nights (or days, hours, minutes, etc.) that the user should wear a device (or devices) at each resistance level. In some variations, a single (e.g., disposable) device may be included for each night that that it should be worn. For example, the user may be instructed to wear the first device (or a device from the set of devices) and each subsequent set of devices for 3 days, in order for them to acclimate to the increasing expiratory resistance level. In another example, the system or kit may just include a series of sequentially labeled devices (or pairs of device if packaged as single-nostril devices) that indicate for each consecutive night which device should be worn; sequentially numbered device may have the same expiratory resistance or the expiratory resistance may increase or decrease slightly, depending on the acclimation strategy.

Thus, described herein are systems for acclimating a subject to a nasal device having a greater expiratory resistance than inspiratory resistance comprising a plurality of nasal devices having increasing resistances to exhalation, wherein most (if not all) of the devices have a resistance to exhalation that is greater than the resistance to inhalation. The plurality of devices are either marked or arranged to indicate the increasing resistance to exhalation corresponding to the order in which the devices are to be used by a subject. These nasal devices typically include an airflow resistor and holdfast, as described herein.

Also described herein are adjustable respiratory devices configured for remote adjustment. For example, any of the variations described above may include a receiver for receiving remote adjustment instructions, and an actuator for modifying the resistance of the device based on the adjustment instructions. For example, the device may include a wireless receiver and actuator (e.g., motor, driver, etc.) configured to modify the expiratory resistance. In some variation the expiratory resistance of the respiratory device may be adjusted by the application of an external magnetic field that acts on the device (e.g., to magnetically move the adjustment member to open/close a leak pathway).

The general principles, and at least some of the variations described above are illustrated in greater detail and described briefly below.

### INCORPORATION BY REFERENCE

All publications and patent applications mentioned in this specification are herein incorporated by reference in their entirety, as if each individual publication or patent application was specifically and individually indicated to be incorporated by reference in its entirety.

### BRIEF DESCRIPTION OF THE DRAWINGS

FIGS. 1A and 1B are bottom and top perspective views, respectively, of one variation of a nasal device.

FIGS. 2A and 2B show one variation of a layered nasal device in a top view and an exploded perspective view, respectively.

FIGS. 3A-3G show variations of nasal devices or portions of nasal devices which may be adapted to be adjustable resistance nasal devices. In particular, FIG. 3A show a whole-nose nasal device that includes conformable holdfasts for insertion into a subject's nostrils. FIG. 3B shows the airflow resistor portion of a nasal device including a relatively stiff flap valve including a central leak pathway. FIG. 3C shows another variation of the airflow resistor including a leak pathway. FIG. 3D illustrates a layered-type nasal device including a flap valve layer, an adhesive holdfast layer, and a protective backing. FIGS. 3E and 3F show whole-nose nasal devices. FIG. 3G is an adhesive nasal device configured to communicate with a single nostril.

FIG. 4A shows a portion of a nasal device, including four leak pathways, and FIG. 4B is a snap-on resistance modifying member.

FIG. 5 is a whole-nose nasal device including removable adhesive covers for adjusting the resistance.

FIG. 6 illustrates a constrictable leak pathway that may be included as part of a nasal device for adjusting the resistance.

FIG. 7 is one variation of an adjustable resistance nasal device in which the airflow resistor is movable to adjust the resistance.

FIGS. 8A and 8B show another variation of an adjustable resistance nasal device in which the airflow resistor is movable to adjust the resistance.

FIG. 9 is a variation of an adjustable resistance nasal device including a movable flap valve and configured so that moving the flap valve alters the resistance.

FIG. 10A shows one variation of an adjustable resistance nasal device in which the valve body is rotatable to adjust the resistance.

FIG. 10B is another variation of an adjustable resistance nasal device in which the flap valve is rotatable relative to the rest of the nasal device to adjust the resistance.

FIG. 11 shows a cross-section through another variation of a nasal device having an adjustable resistance in which the flap valve may be displaced to regulate the expiatory resistance.

FIGS. 12A and 12B show top and side views of one variation of a snap-on device for adjusting the resistance of a nasal device by partially displacing the airflow resistor of the nasal device.

FIG. 12C illustrates operation of device such as that shown in FIGS. 12A and 12B.

FIGS. 13A and 13B show isometric and cross-sectional views, respectively, of another variation of an adjustable resistance nasal device.

FIG. 14 is a partial cross-section though another variation of an adjustable resistance nasal device, in which the length of the leak pathway may be regulated.

FIG. 15 is a bottom view (showing the non-adhesive side facing away from the patient) of an adjustable resistance variation of a nasal device.

FIG. 16A shows a perspective view of another variation of an adjustable-resistance nasal device. FIG. 16B is an alternative view of another variation of a resistance modifying member of an adjustable-resistance nasal device.

FIG. 17A shows a perspective view of another variation of an adjustable-resistance nasal device. FIG. 17B is an alternative view of another variation of a resistance modifying member of an adjustable-resistance nasal device.

FIG. 18A shows a side view of a portion of an adjustable-resistance nasal device including a valved leak pathway. FIG. 18B schematically illustrates the valved leak pathway of FIG. 18A.

FIGS. 19A and 19B illustrate another variation of a resistance modifying member, configured as a weighted expiratory leak path.

FIG. 20As is a perspective view of one variation of a remotely adjustable adjustable-resistance nasal device. FIG. 20B shows a top view of the same device, and FIG. 20C illustrates a detailed view of the resistance modifying member that is remotely actuated.

FIG. 21A is a front view of another variation of a remotely adjustable adjustable-resistance nasal device. FIG. 21B shows an enlarged and simplified view of another variation of a remotely actuateable resistance modifying member.

### DETAILED DESCRIPTION OF THE INVENTION

Described herein are adjustable-resistance respiratory devices, and particularly nasal respiratory devices having passive airflow resistors and a control or controls to adjust the resistance to exhalation through the device. These devices may be referred to as adjustable-resistance nasal devices or simply adjustable nasal devices. Adjustable resistance nasal devices typically include an airflow resistor configured to inhibit exhalation more than inhalation; a holdfast configured to secure the device in communication with the subject (e.g., with the subject's nose), a leak pathway that is independent of the airflow resistor, and a control configured to adjust the resistance to exhalation through the device. The control may adjust the resistance to exhalation by increasing or decreasing the size, shape and/or number of the leak pathway(s) in the device. In general, the resistance to exhalation may be adjusted within the range of between about 10 cm H₂O*sec/L and about 250 cm H₂O*sec/L (e.g., 0.01 and about 0.25 cm H₂O/(ml/sec)) when measured at 100 ml/sec.

As used herein, the singular forms “a,” “an,” and “the” include plural reference unless the context clearly dictates otherwise.

The adjustable resistance features described herein may be used with any appropriate nasal devices, particularly those having a flap valve, including nasal devices for use with known PAP (e.g., biPAP, CPAP, etc.) masks.

### Nasal Devices

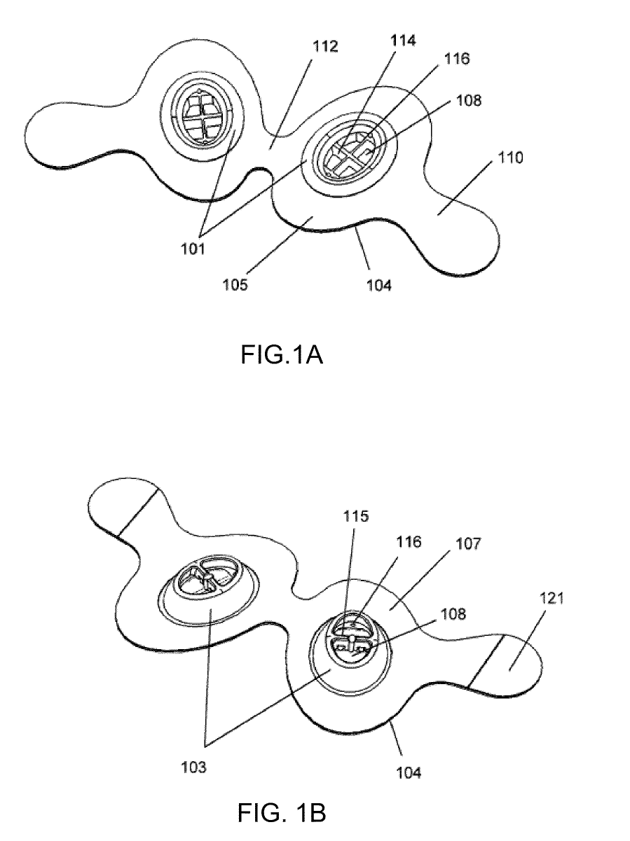

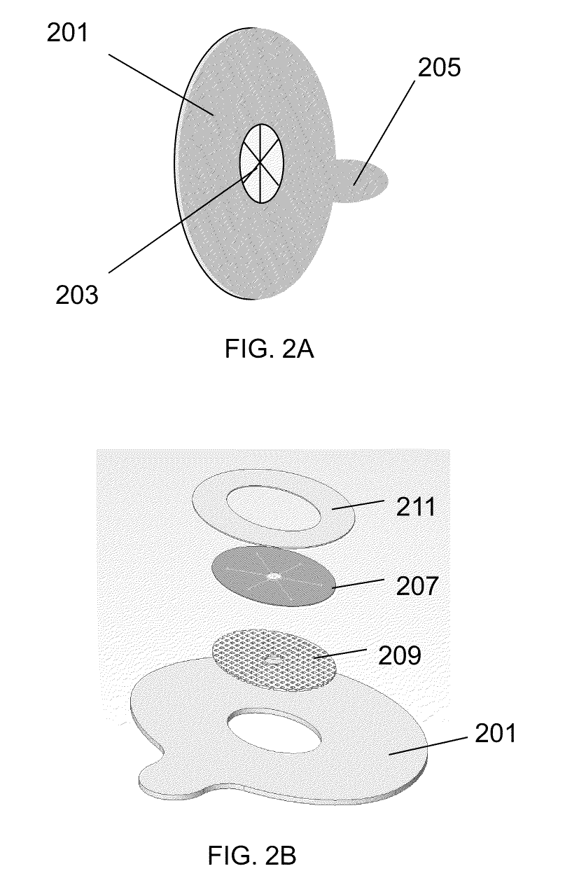

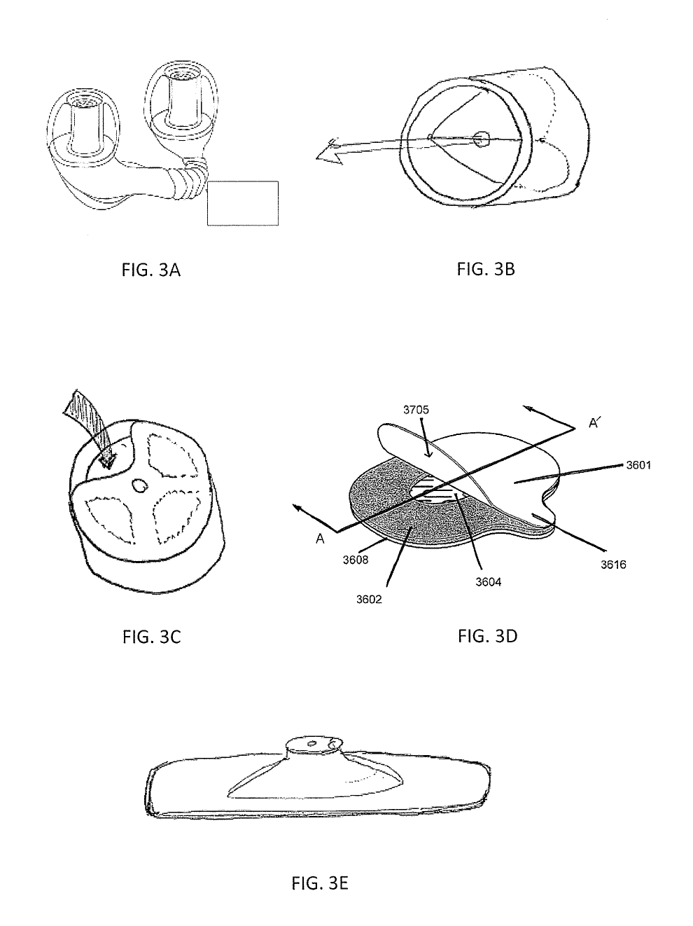

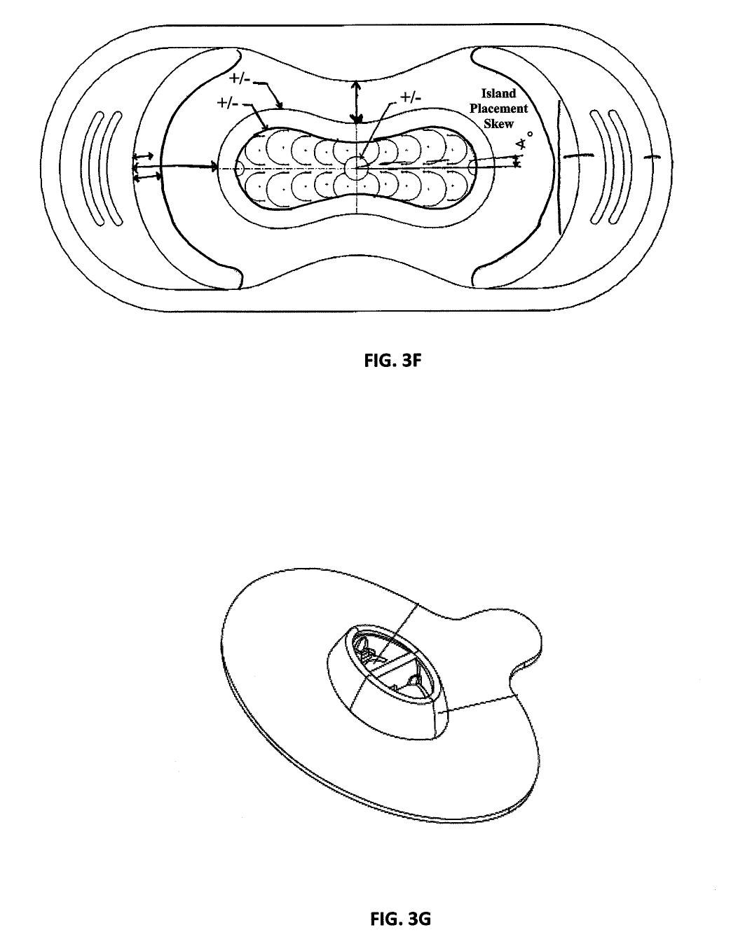

Any appropriate nasal device may be configured as an adjustable-resistance nasal device, including the adhesive nasal devices described in more detail in FIGS. 1A to 2B. The adjustable-resistance nasal devices described herein typically include an opening and/or a passageway configured to communicate with a subject's nasal passage (or cavity), and an airflow resistor in communication with the passageway, and a noise-reduction feature. The airflow resistor may be a flap valve type airflow resistor.

The adjustable-resistance nasal devices described herein may be secured in communication with a subject's nose or nostrils, and specifically with one or both of the subject's nasal cavities. A typical nasal device includes an airflow resistor that is configured to resist airflow in a first direction more than airflow in a second direction, and may also include a holdfast configured to secure the airflow resistor at least partially over, in, and/or across the subject's nose or nostril. The holdfast may include a biocompatible adhesive and a flexible region configured to conform to at least a portion of a subject's nose. The nasal devices described herein are predominantly adhesive nasal devices, however the adjustable-resistance features described may be used with nasal devices that are not adhesive nasal devices, including nasal devices having compressible or expandable holdfasts. Other embodiments include nasal devices in which the holdfast is mask that fits over the nose, the mouth or both the nose and mouth.

Nasal devices may be worn by a subject to modify the airflow thorough one or (more typically) both nostrils. Nasal devices may be secured over both of a subject's nostrils so that airflow through the nostrils passes primarily or exclusively through the nasal device(s). Adhesive nasal devices are removably secured over, partly over, and/or at least partly within the subject's nostrils by an adhesive. The nasal devices described herein may be completely flexible, or partially rigid, or completely rigid. For example, the devices described herein may include an adhesive holdfast region that is at least partially flexible, and an airflow resistor. The airflow resistor may be flexible, or rigid. In some variations, the devices described herein also include one or more alignment guides for helping a subject to orient the device when securing it over the subject's nose. The alignment guide may also include or be configured as a noise-reduction element, as described in greater detail below. The adhesive nasal devices described herein may be composed of layers. Nasal devices composed of layers, which may also be referred to as layered nasal devices, may be completely or partially flexible, as previously mentioned. For example, a layered nasal device may include an airflow resistor configured to resist airflow in a first direction more than airflow in a second direction and an adhesive holdfast layer. In some variations, the airflow resistor may be a flap valve layer adjacent to a flap valve limiting layer, and may include an adhesive holdfast layer comprising an opening across which the airflow resistor is operably secured. The airflow resistor may be disposed substantially in the plane of the adhesive holdfast layer. The adhesive holdfast layer may be made of a flexible substrate that includes an additional layer of biocompatible adhesive.

The nasal devices described herein may be considered as passive nasal devices, because the flap valve may operate to passively regulate a subject's respiration. For example, a nasal device may create positive end expiratory pressure (“PEEP”) and/or expiratory positive airway pressure (“EPAP”) during respiration in a subject wearing the device. In contrast to active nasal devices, such as CPAP machines that apply positive pressure to the subject, the passive devices described herein do not require the addition of pressurized respiratory gas.

The adjustable-resistance nasal devices and methods described herein may be useful to treat a variety of medical conditions, and may also be useful for non-therapeutic purposes. For example, a nasal respiratory device may be used to treat sleep disordered breathing or snoring. The systems, devices and methods described herein are not limited to the particular nasal device embodiments described. Variations of the embodiments described may be made and still fall within the scope of the disclosure.

As used herein, a nasal device may be configured to fit across, partly across, at least partly within, in, over and/or around a single nostril (e.g., a “single-nostril nasal device”), or across, in, over, and/or around both nostrils (“whole-nose nasal device”). Any of the features described for single-nostril nasal devices may be used with whole-nose nasal devices, and vice-versa. In some variations, a nasal device is formed from two single-nostril nasal devices that are connected to form a unitary adhesive nasal device that can be applied to the subject's nose. Single-nostril nasal devices may be connected by a bridge (or bridge region, which may also be referred to as a connector). The bridge may be movable (e.g., flexible), so that the adhesive nasal device may be adjusted to fit a variety of physiognomies. The bridge may be integral to the nasal devices. In some variations, single-nostril nasal devices are used that are not connected by a bridge, but each include an adhesive region, so that (when worn by a user) the adhesive holdfast regions may overlap on the subject's nose.

One variation of an adjustable-resistance nasal device may include a noise-reduction feature (e.g., a noise-reduction flap or noise-reduction element). Other modifications, including sensors for detecting and/or reporting airflow through the nasal device or pressure within the user's nose, may also be included.

In some variations, the adjustable-resistance nasal devices are layered nasal device, formed of two or more layers. For example, a layered nasal device may include an adhesive holdfast layer and an airflow resistor layer. These layers may themselves be composed of separate layers, and these layers may be separated by other layers, or they may be adjacent. An adhesive holdfast layer may be formed of layers (optionally: a substrate layer, a protective covering layer, an adhesive layer, etc), and thus may be referred to as a layered adhesive holdfast. Similarly, the airflow resistor may be formed of multiple layers (optionally: a flap valve layer, a valve limiter layer, etc.), and thus may be referred to as a layered airflow resistor. In some variations, the layered adhesive holdfast and the layered airflow resistor share one or more layers. For example, the flap valves layer and the adhesive substrate layer may be the same layer, in which the leaflets of the flap valve layer are cut from the substrate layer material. As used herein, a “layer” may be a structure having a generally planar geometry (e.g., flat), although it may have a thickness, which may be uniform or non-uniform in section. As mentioned briefly above, the support backing may be formed of one of the layers of a layered nasal device, such as the adhesive substrate layer.

In some variations, an adjustable-resistance nasal device has a body region including a passageway configured to be placed in communication with a subject's nasal passage. The body region may be a stiff or flexible body region, and may secure an airflow resistor therein. In some variations, the body region is at least partially surrounded by a holdfast (i.e., a planar adhesive holdfast). The body region may be modular, meaning that it is formed of two or more component sections that are joined together. Examples of such nasal devices can be found in U.S. Pat. No. 7,506,649, filed on Jun. 7, 2007, and previously incorporated by reference in its entirety. As described therein, the body region may be configured so that it does not irritate a subject wearing the nasal device. For example, the body region may be slightly undersized compared to the size of the average user's nostrils. Thus the body region may fit into the subject's nose, and the seal with the subject's nose is formed by the adhesive holdfast region, rather than the body region. In some variations the body region does not substantially contact the inner walls of the subject's nose. Furthermore, the body region may extend only slightly into the subject's nose.

In some variations, the adhesive nasal device includes a support frame. The support frame may provide structural support to all or a portion of the nasal device, such as the flexible adhesive portion. For example, the support frame may support the adhesive holdfast portion of the device and be completely or partially removable after the device has been applied to the subject. In some variations, the support frame remains on the nasal device after application. In some variations, the support frame is a support frame layer.

An adjustable-resistance adhesive nasal device may also include a tab or handle configured to be grasped by a subject applying the device. In some variations, this tab or handle is formed of a region of the layered adhesive holdfast.

The various components of the device may be made of any appropriate materials, as described in greater detail below. For example, some device components (e.g., an alignment guide, a body region, noise-reduction element, control, resistance modifying member) may be made of medical grade plastic, such as Acrylonitrile Butadiene Styrene (ABS), polypropylene, polyethylene, polycarbonate, polyurethane or polyetheretherketone. The airflow resistor may be a flap valve and the flap may be made of silicone or thermoplastic urethane. The adhesive holdfast may include an adhesive substrate made of silicone, polyurethane or polyethylene. Examples of biocompatible adhesive on the adhesive holdfast may include hydrocolloids or acrylics. These lists of materials are not exclusive, and other (or alternative) materials may be used.

In some versions, the nasal device further comprises an active agent. In some versions, this active agent is a drug (e.g., a medicament). In some versions, this active agent comprises an odorant, such as a fragrance. In some versions, the active agent comprises menthol, eucalyptus oil, and/or phenol. In other versions, the nasal device may be used with other pulmonary or medical devices that can administer medication or other medical treatment, including, but not limited to, inhalers and nebulizers.

A nasal device may include a filter. This filter may be a movable filter, such as a filter that filters air flowing through the passageway in one direction more than another direction (e.g., the device may filter during inhalation but not exhalation).

As mentioned, the adjustable-resistance nasal devices described herein typically include a holdfast region (or layer) and at least one airflow resistor. As will be apparent from the figures, many of these nasal devices may be removable and insertable by a user without special tools. In some variations, a subject may use an applicator to apply the device (e.g., to help align it). FIGS. 1A through 2B illustrate different exemplary nasal devices.

FIGS. 1A and 1B show perspective views of one exemplary variation of an adhesive nasal device that may be configured as an adjustable-resistance nasal device. FIG. 1A shows a front perspective view of an adhesive nasal device, looking at the “outer” side of the device, which is the side facing away from the subject's nose when the device is worn. The device shown in FIG. 1A includes two single-nostril rim bodies 101 and a single adhesive holdfast 104. A nasal device may be configured to communicate with a single nostril (a single-nostril nasal device), or it may be configured to communicate with both of a subject's nostrils (a double-nostril nasal device as shown here).

The holdfast 104 (which adhesively secures the device to the subject) is shown as a layered structure including a backing or adhesive substrate 105. This backing may act as a substrate for an adhesive material, or it may itself be adhesive. The holdfast 104 may have different regions, including two peri-nasal regions surrounding the rim bodies 101. Each rim body has at least one passageway 108 for airflow therethrough. The adhesive holdfast also includes two tabs or grip regions 110 that may make the device easier to grasp, apply, and remove. A bridge region 112 is also shown. In this example, the bridge region is part of the adhesive holdfast (e.g., is formed by the same substrate of the adhesive holdfast) and connects the peri-nasal regions. Although the tab and bridge regions are shown as being formed as part of (integral with) the holdfast material, these regions may also be formed separately, and may be made of different materials.

The rim body regions 101 shown in the exemplary device of FIG. 1A include outer rim body regions which each encompass a passageway 108. These first (e.g., outer) rim body regions may mate with second (e.g., inner) rim body regions to form the rim body region(s) of the device that each include a passageway 108. This passageway is interrupted by crossing support members 114 (e.g., cross-beams or cross-struts) that may partly support or restrict movement of the airflow restrictor. In addition, each rim body region 101 includes two leak pathways 116, through which air may pass even when the passageway through the device is otherwise blocked by the airflow resistors. The leak pathways 116 are shown here as small openings at the narrow ends of the oval-shaped outer rim body region. The rim body region may also be referred to as ‘rim’ or ‘scaffold’ regions of the device. The rim bodies in this example are separate from the airflow resistor, and particularly the moving portion of the airflow resistor, such as a flap valve. Thus, the leak pathways 116 may be referred to as ‘isolated’ from the airflow resistor or the moving portion of the airflow resistor. In some variations a leak pathway may be formed through the airflow resistor (e.g., flap valve) or around it.

FIG. 1B shows a back perspective view of the opposite side of the adhesive nasal device shown in FIG. 1A, the “inner side” of the device. The inner side of the device faces the subject, and a portion of this side of the device may contact the subject. This side of the device, and particularly the adhesive holdfast of the device, includes an adhesive (which may be covered by a protective cover 107) forming part of the holdfast 104. In some variations, the entire skin-facing side of the holdfast 104 includes an adhesive on the surface, although in some variations, only a portion of this region includes adhesive. The adhesive may be a distinct layer of the holdfast (e.g., it may be layered on top of an adhesive substrate), or it may be an integral part of the holdfast (e.g., the adhesive substrate may be made of an adhesive material). In some variations an adhesive may be separately added to the device (e.g., the holdfast region) before use. The adhesive material may be covered by a removable protective cover or liner 107, to prevent the adhesive from sticking to surfaces until after the liner is removed. In FIG. 1B, the protective cover 107 covers the entire skin-facing surface of the holdfast. The device may be applied by first removing the liner. For example, the liner may be peeled off, to expose the adhesive. In some variations, the liner protecting the adhesive may be partially removed. For example, the tab region 121 of the device may include a separate (or additional) liner that remains over the tab region when other liner regions are removed. This may allow the device to be held by the tab region without having it adhere to the skin. After removing the cover, or a part of the cover, the device may be positioned and adhered to the subject's skin around the nasal cavity, so that the passageways through the rim body are aligned with the openings of the subject's nasal cavities. In some variations, an additional adhesive cover region (e.g., the protective cover region over the tabs 121) can then be removed to secure the device to the rest of the subject's nose. The adhesive cover may include a fold (or crimp, crease, lip, or the like) that helps to remove the protective cover from the adhesive.

The second, or inner, rim body region 103 shown in the exemplary device of FIG. 1B is shaped with an inwardly-tapering edge, so that it may fit at least slightly within the opening of the subject's nostril when a subject wears the device. The inner rim body includes one or more passageways 108 that correspond with the passageways 108 shown in FIG. 1A. Similarly, the leak pathways pass completely through the rim body (both inner and outer bodies). The tapering external walls of the inner rim body region(s) shown in FIG. 1B are shown as smooth, and may also include an additional material (e.g., an auxiliary holdfast material) for securing them in the subject's nostrils, or for cushioning them to prevent injury or discomfort. These surfaces may also be more or less angled, in order to facilitate comfort when the adhesive nasal device is worn in the subject's nose. A cross bar (hinge region 115) may also be provided as part of the inner rim body. The inner rim body 103 may extend some distance above the peri-nasal annular region of the holdfast, as shown in FIG. 1B. This distance may be sufficient to prevent any portion of the airflow resistor (e.g., a flap portion of a flap valve) from extending out of the device and into the nasal cavity where it might contact body tissues (which may interfere with its operation).

All of the nasal devices described herein also include an airflow resistor, which is located in one or more passageways formed through the device. In FIGS. 1A and 1B, the airflow resistor is a flap valve, and includes cross bars that support the flap valve (and can prevent it from opening during exhalation). In general, the airflow resistor opens in one direction (during inhalation) and is closed during exhalation. The flap may be made of silicone. In the device shown in FIGS. 1A and 1B, the flap can be secured between the inner and outer rim bodies.

FIG. 2A is a top view of another example of a nasal device. The nasal device shown in FIGS. 2A-2B is a layered nasal device that includes a holdfast layer 201 and an airflow resistor 203. The reverse side of the device shown in FIG. 2A includes an adhesive material (not shown) that may be covered by a protective covering. The protective covering (which may also be referred to as a protective liner) can be removed to expose the adhesive before application of the device. Thus, the holdfast layer of the device secures it to the subject. This holdfast layer may itself be layered, and may include an adhesive substrate (e.g., a backing layer). For example, the adhesive substrate may be a foam backing. This backing may act as a substrate for an adhesive material. In some variations, the adhesive substrate is itself adhesive. The holdfast layer 201 may have different regions, including a peri-nasal regions surrounding a passageway (though which air may flow), and a tab 205 or grip region forming a tab that may make the device easier to grasp, apply and remove. Other regions may include regions of more aggressive and less aggressive adhesive (e.g., more or less adhesive material), or regions of hydrogel material (including adhesive hydrogels) to help prevent irritation from repeated or extended use. Although the tab is shown as part of (integral with) the holdfast material, this region may also be formed separately, and may be made of different materials.

FIG. 2B shows an exploded view of the device of FIG. 2A. This exploded perspective view illustrates the layers of the device, including the adhesive holdfast 201 (which may itself be layered), two layers forming the airflow resistor, including the flap valve 207 and flap valve limiter 209, and an adhesive ring 211 that may help attach the flap valve and flap valve limiter to the adhesive holdfast.

An adhesive holdfast for a nasal device may comprise any appropriate material. For example, the adhesive substrate may be a biocompatible material such as silicone, polyethylene, or polyethylene foam. Other appropriate biocompatible materials may include some of the materials previously described, such as biocompatible polymers and/or elastomers. Suitable biocompatible polymers may include materials such as: a homopolymer and copolymers of vinyl acetate (such as ethylene vinyl acetate copolymer and polyvinylchloride copolymers), a homopolymer and copolymers of acrylates (such as polypropylene, polymethylmethacrylate, polyethylmethacrylate, polymethacrylate, ethylene glycol dimethacrylate, ethylene dimethacrylate and hydroxymethyl methacrylate, and the like), polyvinylpyrrolidone, 2-pyrrolidone, polyacrylonitrile butadiene, polyamides, fluoropolymers (such as polytetrafluoroethylene and polyvinyl fluoride), a homopolymer and copolymers of styrene acrylonitrile, cellulose acetate, a homopolymer and copolymers of acrylonitrile butadiene styrene, polymethylpentene, polysulfones polyimides, polyisobutylene, polymethylstyrene and other similar compounds known to those skilled in the art. Structurally, the substrate may be a film, foil, woven, non-woven, foam, or tissue material (e.g., poluelofin non-woven materials, polyurethane woven materials, polyethylene foams, polyurethane foams, polyurethane film, etc.).

In variations in which an adhesive is applied to the substrate, the adhesive may comprise a medical grade adhesive such as a hydrocolloid or an acrylic. Medical grade adhesives may include foamed adhesives, acrylic co-polymer adhesives, porous acrylics, synthetic rubber-based adhesives, silicone adhesive formulations (e.g., silicone gel adhesive), and absorbent hydrocolloids and hydrogels.

Adjustable-resistance nasal devices may be worn to treat any disorder that would benefit from the use of a nasal device, including but not limited to respiratory or sleeping disorders, such as snoring, sleep apnea (obstructive, central, mixed and complex), COPD, cystic fibrosis and the like. Adjustable-resistance nasal device may be particularly beneficial for treatments in which the subject is encouraged or permitted to sleep while wearing the device, because they may be adjusted to allow for comfortable and/or therapeutic use. The adjustable-resistance features of these nasal devices may be used to optimize the effect (e.g., the resistance to exhalation) applied by the device. To use the adjustable-resistance nasal device, it is first placed in communication with the subject's nasal cavity so that airflow from the subject's nose passes through the device as it is worn. In some variations, the resistance is set/adjusted prior to application of the device. The adjustable-resistance feature (e.g., a control and/or a resistance modifying member) may then be used to adjust the expiratory resistance through the device. The nasal device may be placed in communication with the nasal passageway by placing it into or at least partially over or around the subject's nasal cavity. For example, an adhesive holdfast attached to the nasal device may be used to secure the device in position.

Many other materials and structures may be used to achieve the adjustable-resistance features described. This description is not intended to be limited to the structures and materials mentioned, but is intended to also encompass many other materials and structures having similar properties.

In some variations, an adjustable resistance nasal device includes an airflow resistor configured to inhibit exhalation more than inhalation, a holdfast configured to secure the nasal device in communication with a subject's nostril, a leak pathway through the nasal device that is separate from the airflow resistor and is configured to be open during both exhalation and inhalation, and a control configured to adjust the expiratory resistance through the nasal device. The control may be a remote control that can be adjusted by a third party (e.g., physician, technician, sleep partner, or the like) to change the expiratory resistance, or it may be a control that can be manipulated by the wearer or user of the device, or both. For example, a control may include a knob, a dial, a button, a tab, a lever, a pin, a pull cord, a pull tab, or the like. In some variations, the adjustable resistance nasal device includes a resistance modifying member that is configured to modify the leak pathway to adjust the expiratory resistance.

For example, described herein are adjustable resistance nasal devices including: an airflow resistor configured to inhibit exhalation more than inhalation; a holdfast configured to secure the nasal device in communication with a subject's nasal cavity; a leak pathway through the nasal device that is separate from the airflow resistor and configured to be open during both exhalation and inhalation; and a resistance modifying member configured to allow adjustment of airflow through the leak pathway. The resistance modifying member may be coupled to (and controlled by) a controller, as mentioned above.

A resistance modifying member is generally configured to modify the expiratory resistance through the nasal device. For example, the resistance modifying member may change the shape, size (e.g., diameter, length, etc.) and/or number of leak pathways. For example, a resistance modifying member may include a cover or shutter that adjustably occludes all or a portion of a leak pathway. In some variations, the resistance modifying member is an adjustable valve, such as a needle valve or a weighted valve, that can be adjusted to alter the expiratory resistance through the leak path(s) of the nasal device. In some variations, the leak pathway is adjustable. For example, the leak pathway may be constrictable or dilatable to open/close to some degree, thereby decreasing/increasing the expiratory resistance.

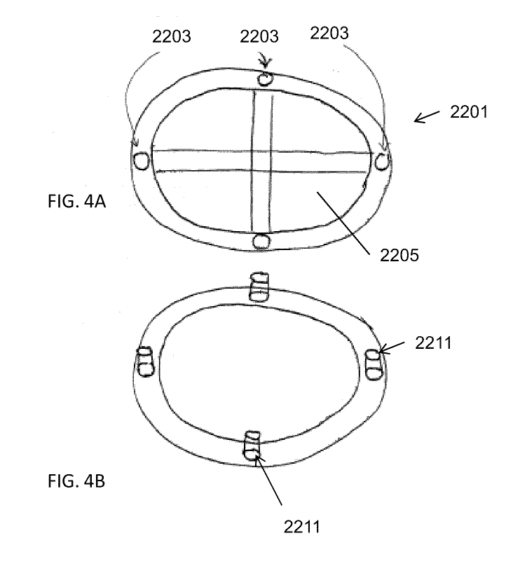

FIGS. 4A through 21A illustrate different variations of adjustable-resistance nasal devices and method of using them, as well as resistance modifying members that may be used with nasal devices to form adjustable-resistance nasal devices. A resistance-modifying nasal device may include, for example: a resistance modifying member such as a plug or cover that blocks one or more leak pathways, an adjustable cover (such as a shutter including louvers/sliders to cover all or a portion of the leak pathway), and an adjustable valve to increase/decrease the size of the leak pathway. In some variations, the expiratory resistance of a nasal device is adjustable using an adjustable airflow resistor that may be adjusted to prevent a complete seal by the edges and/or the center of the airflow resistor when the device is worn; one or more constrictable holes; and one or more leak pathways whose length can be changed to increase/decrease the resistance.

As mentioned, in some variations of the adjustable-resistance nasal devices described herein, a nasal device may be adjustable by covering or blocking a leak pathway. The leak pathway (typically a pre-formed leak pathway on any appropriate portion of the nasal device that is separate from the airflow resistor) may be completely or partially covered in a controllable fashion. For example a nasal device may be used with a resistance-modifying member such as that shown in FIG. 4B. FIG. 4A shows a portion of a nasal device 2201 including four leak pathways 2203 which are openings around the perimeter of a valved passageway 2205 (the valve is not shown in FIG. 4A). These leak pathways may be open during both inspiration and exhalation. The expiratory resistance may be modified by plugging any of these leak pathways, thereby increasing expiratory resistance, or by unplugging them, thereby decreasing expiratory resistance. For example, FIG. 4B illustrates one variation of a resistance-modifying member that includes plugs 2211 that may be used to block these leak pathways. In this variations, the resistance-modifying member is a snap-on device that may be attached (e.g., friction fit) to the nasal device to block one or more of the leak pathways. The device shown in FIG. 4B includes four plugs, however variations in having more or fewer plugs may be used. The plugs may be partial plugs, so that the diameter of the leak pathway may be reduced by some percentage (e.g., 10%, 20%, 25%, 50%, 75%, etc.) to increase resistance. In some variations, the ‘plug’ portions of the snap-on device are removable or adjustable. For example, the “plug” may be a slider or shutter that can be moved across the leak pathway(s) to partially occlude them. Thus, virtually any nasal device may be adapted to be a variable-resistance nasal device by including an attachable resistance-modifying member. In some variations the resistance-modifying member does not occlude or otherwise block the valved central opening, and therefore it does not modify inspiratory resistance. Although the resistance-modifying member shown in FIG. 4B is a snap-on resistance-modifying member, a resistance-modifying member may be attached to the nasal device in other ways as well. For example, the resistance-modifying member may be adhesively secured to the nasal device, magnetically secured to the nasal device, etc.

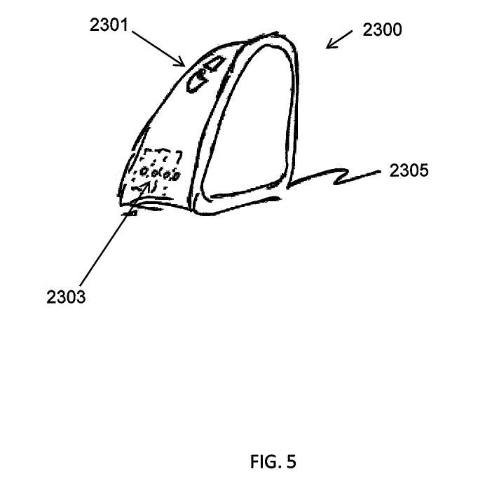

FIG. 5 illustrates another variation of an adjustable-resistance nasal device including a plug or cover which may occlude or partially occlude one or more of the leak pathways in the nasal device. In this example, the nasal device 2300 is a whole-nose nasal device that may fit over the subject's nose, and includes an airflow resistor (e.g., flap valve) 2301, and a plurality of openings (leak pathways) 2303 that may be covered with an adhesive tape or plug. This device may be adhesively secured to a subject's nose by an adhesive holdfast 2305 or other holdfast. Alternatively, the holdfast may not comprise adhesive. In some embodiments, the whole-nose nasal device will be a nose mask that is roughly the shape of a user's nose (whether customized or not) and may be held in place using a temporary adhesive, straps, tethers or the like. The mask is designed to create a complete or partial seal with the user's nose or face. A soft interface material (e.g. silicone or foam for example) may be used to promote a seal and provide user comfort. The mask may be reusable to single-use. In some embodiments, the whole-nose nasal device can be configured for use with active positive airway pressure devices including CPAP, Bi-level PAP, VPAP and the like.

In some variations of the adjustable-resistance nasal devices described herein, the plugs or covers may be integrated into the nasal device, without the need for a separate resistance-modifying member. For example, a nasal device may include a cover or plug that integral with the nasal device or linked to the nasal device (e.g., by a hinge or tether).

Other variations of adjustable-resistance nasal devices may include adjustable leak pathways. For example, a leak pathway may be constrictable, so that the cross-sectional diameter of the leak pathway may be decreased or increased. In some variations, the leak pathway includes a diaphragm, shutter or other member that may be used to expand or constrict the opening of the leak pathway. For example, the leak pathway may include a louver-type cover which can be opened or closed to various degrees. A leak pathway may include a dilating iris-type shutter which can be closed to increase resistance. In some variations the leak pathway includes an inflatable or swellable material to reduce the diameter of the leak pathway. A control that may be used to open/close the constrictable leak pathway may also be included on the nasal device. For example, the control may be a dial, button, slider, or the like.

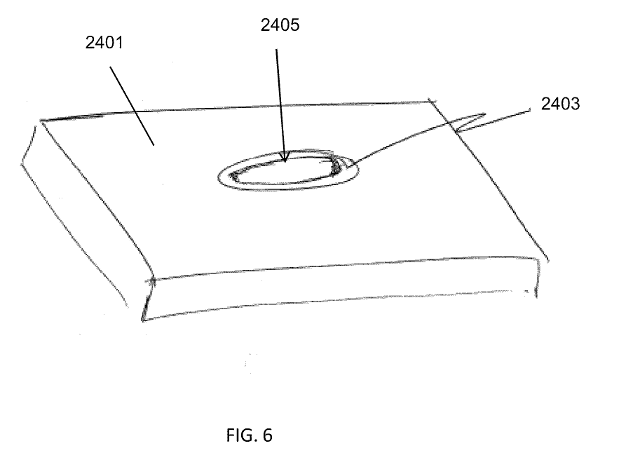

In some embodiments, a porous material including but not limited to some formulations of polyethylene or polypropylene (such as Porex® brand products) may find use. These porous plastics have pores that can become filled with condensed water vapor. When such porous materials are used in any of the components of the devices described herein (including the holdfast or rim), the resistance through the device will adjust or increase as the user breathes through the device, as the pores are plugged or filled and therefore resistance will increase in time. For example, FIG. 6 shows one variation of a constrictable leak pathway 2405, in which the diameter of the leak pathway may be increased or decreased. FIG. 6 shows a magnified view of a single leak pathway which may be part of a nasal device 2401. In some variations, an adjustable nasal device includes a leak pathway 2405 having the wall (or a portion of the wall) 2403 that is inflatable (e.g., an inflatable bladder or plug) that can be inflated to occlude the leak pathway. As mentioned, the leak pathway may include a swellable material that can be swollen to at least partially occlude the leak pathway. The resistance may be adjusted by adding fluid to cause the material to swell and occlude one or more leak pathways.

An adjustable-resistance nasal device may also include an adjustable airflow resistor that may be manipulated to adjust the expiratory (and/or inspiratory) resistance. For example, an adjustable airflow resistor may be moved to modify one or more leak pathways through the device. For example, a nasal device may include an airflow resistor that can be rotated to enlarge or reduce a leak pathway. In some variations the airflow resistor is in communication with a central passageway through the device, and the airflow resistor may be moved in or out of register with the central passageway, creating or eliminating a leak pathway adjacent to the airflow resistor. In some variations, moving the airflow resistor may enlarge or contract a leak pathway formed between the nasal device and the subject wearing the device.

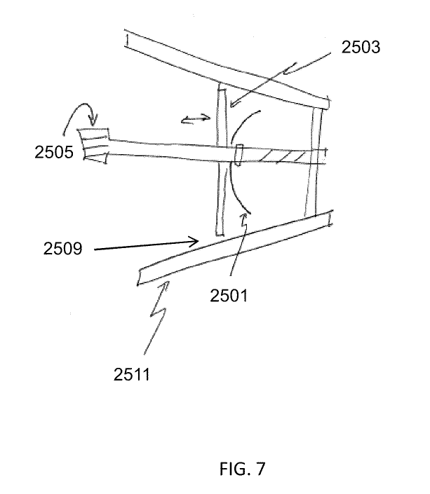

FIG. 7 illustrates one variation of an adjustable-resistance nasal device in which a leak pathway 2509 is formed around the airflow resistor 2501, 2503 as the airflow resistor is moved proximally or distally within a tapered central passageway 2511. The device includes a control knob 2505 that can be turned to move the airflow resistor proximally or distally, to increase or decrease the size of the leak around the device (and thus modify the expiratory resistance when the airflow resistor is otherwise closed). In this example, the airflow resistor includes a flap/diaphragm 2501 and a flap limiter 2503.

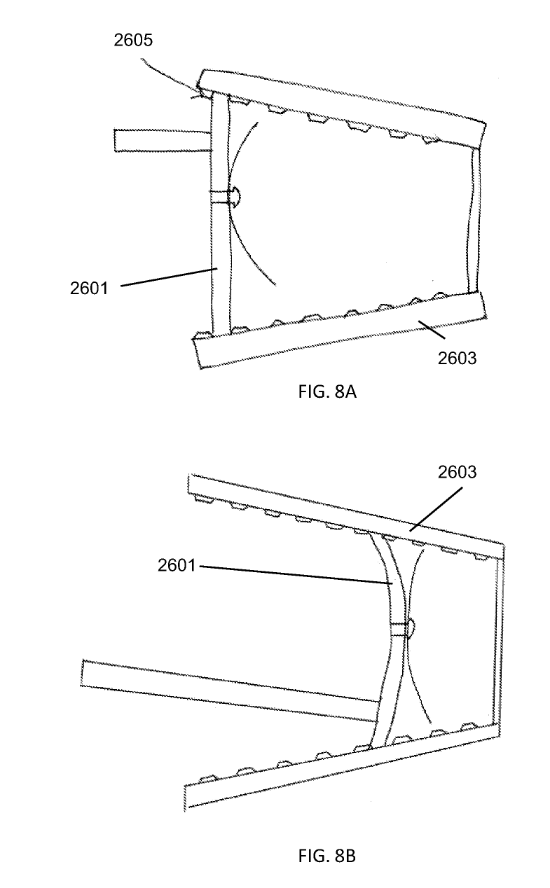

FIGS. 8A and 8B illustrate an alternative variation of an adjustable-resistance nasal device, in which the internal surface of the central passageway 2603 is threaded 2605, and the airflow resistor 2601 may be moved (e.g., by rotating) proximally or distally, causing the airflow resistor to flex. This flexing of the airflow resistor 2601 (and particularly the seating portion for the flap valve) may prevent the valve from closing during exhalation, decreasing the resistance. This embodiment may also provide feedback to the user as the resistance is decreased, since it may become progressively more difficult to advance the airflow resistor proximally (to the right in FIGS. 8A and 8B).

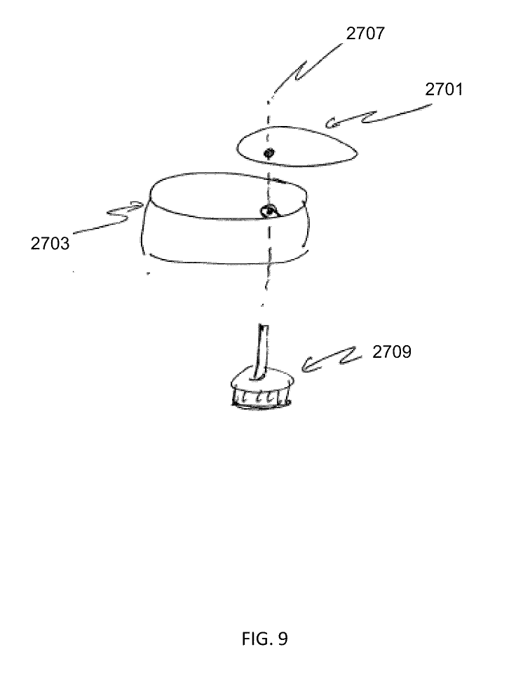

FIG. 9 illustrates another variation of an adjustable-resistance nasal device, in which the flap valve portion 2701 of the airflow resistor may be moved off-center from the central passageway 2703 by turning the knob 2709, rotating the flap of the airflow resistor around a pivot axis 2707, so that a leak pathway may be formed around the flap 2701. Displacing an entire airflow resistor or a portion of an airflow resistor (e.g., the flap portion) may be particularly useful to open and close leak pathways that are not pre-formed but form as the airflow resistor is displaced. In one variation of this concept, the knob may rotate the airflow resistor or a portion of the airflow resistor (e.g., the flap of a flap valve airflow resistor) around a central axis but the airflow resistor or flap of the airflow resistor is moved out of register with the opening or passageway that is regulated by the airflow resistor. For example, if the airflow resistor and passageway are non-circular (e.g., oval).

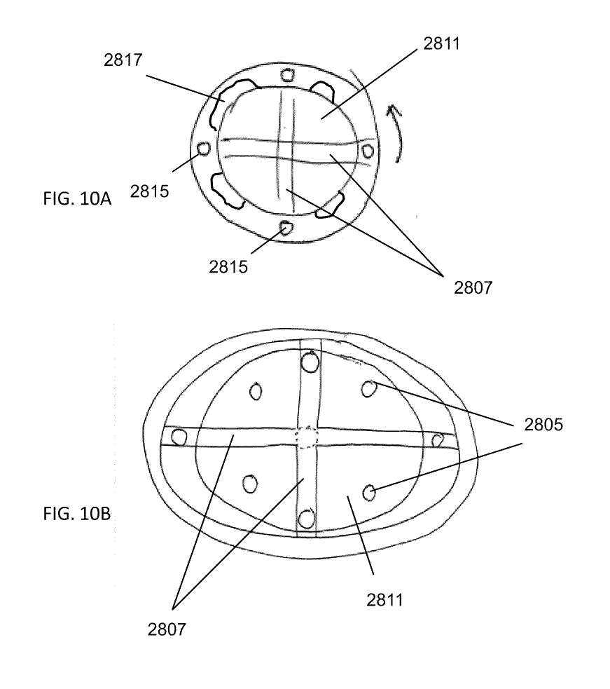

In one alternative embodiment, an example of which is illustrated in FIGS. 10A and 10B, rotation of all or a part of the airflow resistor with respect to the body of the nasal device results in blocking or unblocking pre-formed leak pathways. For example, in FIG. 10A, the flap of the flap valve 2811 is rotatable around the central axis. The edge of the flap 2801 includes projection regions 2817 (which may be different sizes) that may be rotated to cover one or more of the leak pathways (openings 2815) in the region surrounding the central passageway. The flap is shown as transparent in this example, so that the supporting cross-beams 2807 forming the flap valve limiter may be seen.

In FIG. 10B the leak nasal device include six leak pathways 2805 on the surface of the flap valve. The flap valve is supported by two cross-beams forming a “+” pattern on which the flap may sit. These cross-beams are one variation of a flap valve limiter that limits the valve from opening during exhalation. In this example, the flap valve limiter may also block the leak pathway openings through the flap valve when the openings are aligned with the cross-beams 2807. Thus rotation of the flap valve with respect to the cross-beams may expose or cover the leak pathways on the flap valve 2811. In FIG. 10B, the valve is oriented so that four of the leak pathways on the flap (holes 2805) are opened; by rotating the flap 2811, two or four of the holes may be partially or completely blocked when the valve is closed (e.g., during exhalation). Thus, the resistance to exhalation may be adjusted in discrete steps (leak paths unblocked, two leak paths blocked, four blocked, etc.).

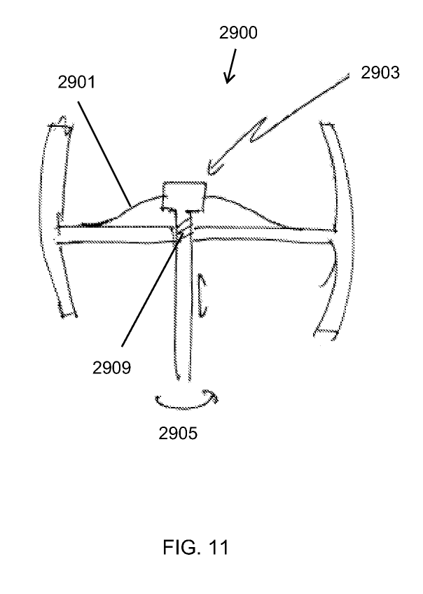

The resistance of an adjustable resistance nasal device may also be adjusted by deflecting all or a portion of the airflow resistor distally or laterally with respect to the passageway through the nasal device, as illustrated in FIGS. 11-14. For example, in FIG. 11, the airflow resistor 2091 of the nasal device 2900 (shown in cross-section) may be displaced up (proximally) so that the flap valve cannot seat on the flap valve limiter (e.g., cross beams), preventing the edges of the flap valve 2901 from sealing and may allow leak flow around the flap, decreasing resistance. The more the flap valve is displaced, the less the resistance. A handle or knob 2905 may be used to displace the flap. In this example, knob is threaded 2909 so that as it is rotated, the flap valve is raised or lowered to increase or decrease the leak pathway and thereby decrease or increase the resistance to exhalation.

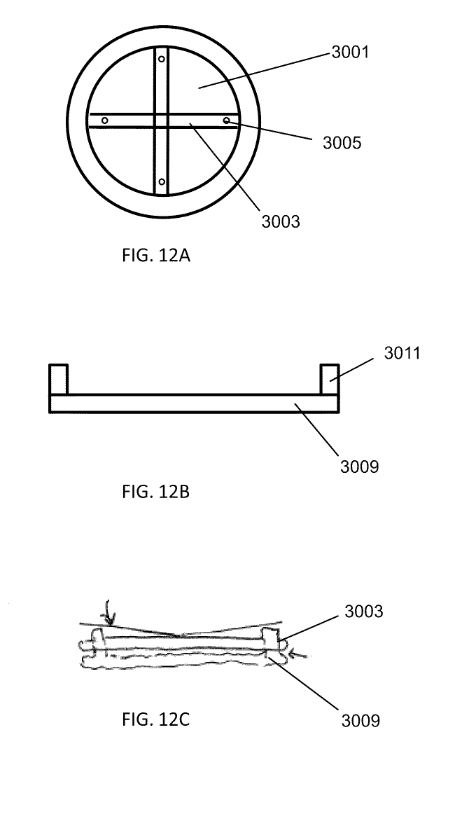

FIGS. 12A and 12B illustrate another variation, in which a resistance-modifying member may be used with a nasal device to displace a portion of the airflow resistor. FIG. 12A shows a bottom view of a nasal device, showing the flap valve 3001 resting against the valve limiting layer (shown as cross-hair beams 3003). The valve limiting layer includes openings 3005, into which a flap valve displacing member 3009 (shown in profile in FIG. 12B) may be inserted. In this example, the resistance-modifying member is the displacing member 3009 which includes four displacing elements 3011 that project from the resistance-modifying member through the openings in the valve limiting layer 3003 to prop open the edges of the flap valve 3001, preventing the flap from closing completely during exhalation, as illustrated in FIG. 12C in partial cross-section.

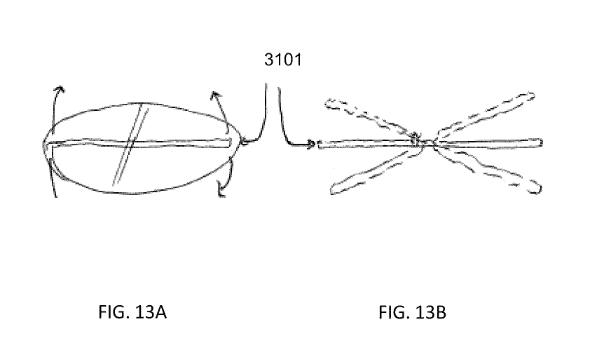

FIGS. 13A and 13B shows another variation of an adjustable-resistance nasal device, in which the nasal device includes a flap valve limiter (cross bars 3101) which may be deflected up or down from the plane of the airflow resistor (when in the ‘closed’ position). For example, the valve limiting member (cross-hairs) may be hinged or bendable so that it can be moved to prevent the nasal device from closing completely, forming a leak pathway around the flap and decreasing expiratory resistance.

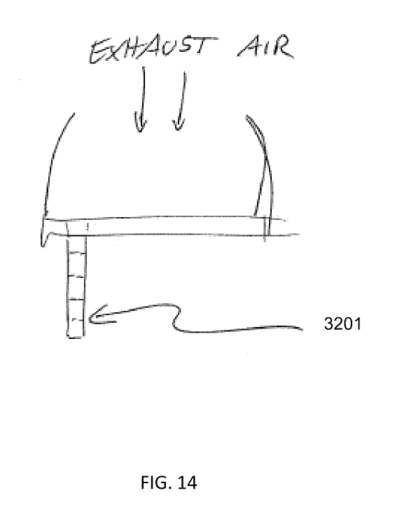

In addition to modifying the position or structure of the airflow resistor to modify resistance, and/or changing the opening size of a leak pathway to modify the resistance, the length of the leak pathway may also be modified to change the resistance. For example, FIG. 14 illustrates one variation in which the length of the leak pathway 3201 may be decreased to decrease the resistance. In this example, the leak pathway is formed by segments that may be removed (or added) to modify the resistance through the leak pathway, and therefore the resistance to exhalation. In some variations the leak pathway may be telescoping in length, so that it can be shortened or lengthened without removing segments.

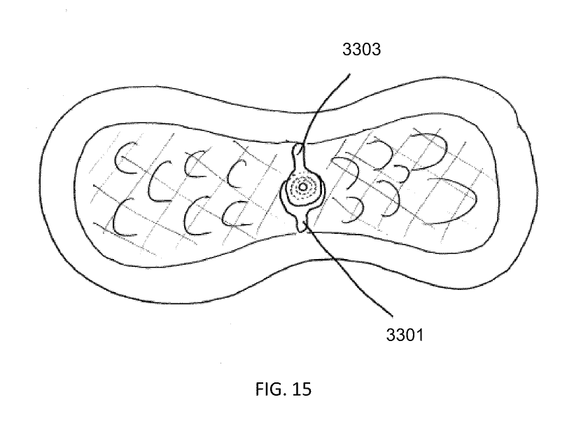

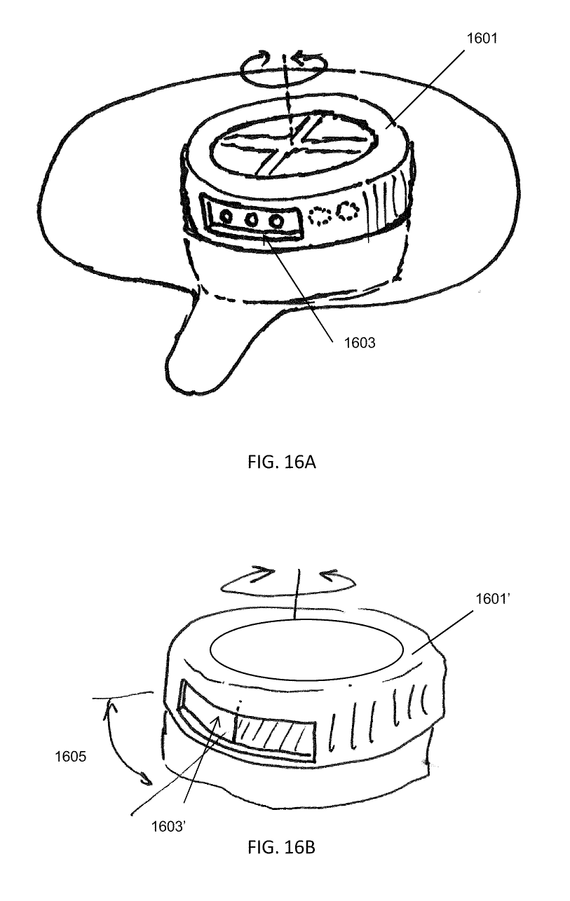

FIG. 15 shows another variation of an adjustable resistance nasal device that includes two adhesively removable resistance modifying members 3301, 3303. In FIG. 15, these resistance modifying members are initially (in this variation) attached to the nasal device so that a central leak pathway through the nasal device is partially occluded. Two separate modifying members 3301, 3303 are layered over the leak pathway, and each other so that the outermost resistance modifying member (removable tab 3303) has a small diameter opening that restricts the leak pathway to this small diameter size. The second resistance modifying member (removable tab 3301) has a slightly larger opening than that of removable tab 3303, but is still slightly smaller than the leak pathway opening of the nasal device. In operation, the first and the second (or both the first and second) resistance modifying members may be removed to progressively decrease the resistance to exhalation. Similarly, adhesive resistance modifying members may be added to partially obstruct the leak pathway, and thereby increase the resistance to exhalation. In this example, the resistance modifying members may include tabs or grasping regions that may be gripped and removed to pull off the adhesive resistance modifying member.

The nasal device shown in FIG. 15 is an adhesive nasal device that is layered and substantially planar, as described above. In this variation, the airflow resistor comprises a layer having a plurality of flap valves that is backed by a valve limiting layer (e.g., mesh). The central leak pathway (hole) is at least partially occluded by the adhesive resistance modifying member, as indicated. Additional leak pathways may also be modified in this way. The perimeter of the device comprises the adhesive holdfast that is configured to adhere (and seal) to the subject's nose.

FIGS. 16A and 16B illustrate another variation of an adjustable resistance nasal device including a manually adjustable resistance modifying member. In FIG. 16A, the nasal device includes a resistance modifying member that is configured as a dial 1601 that can be rotated (e.g., manually or, as described below, automatically). In this example, the dial 1601 is therefore both the resistance modifying member and the adjustable control. The dial 1601 includes a window on the side face that is configured to cover or expose one or more leak pathways 1603 located on a portion of the airflow resistor. In this variation, the leak pathways 1603 are located on a side of a body region of the nasal device. Rotation of the control 1601 dial exposes or covers these leak pathways, thereby changing the expiratory resistance through the device. The resistance modifying member may include indicators (e.g., aural, tactile, etc.) indicating discrete adjustment points For example, the dial may include ‘stops’, detents, or friction points after exposing each sequential leak pathway. These detents may be calibrated to discrete expiratory resistance values, as described above, and may allow discrete, stepped rotation of the dial.

Any of the adjustable resistance nasal devices described herein may include one or more indicators configured to indicate the resistance of the device. In particular, the devices may include one or more indicators that indicate the expiratory resistance (e.g., the resistance to exhalation). For example, an indicator may be a visual indicator, which indicates the resistance to exhalation by an alphanumeric; the indicator may indicate an approximate estimate of the expiratory resistance (as cm H₂O/(ml/sec)) when measured at 100 ml/sec), or it may indicate based on the state of the resistance modifying member. In some variations the various settings of the resistance modifying member may be coordinated with pre-determined (or pre-set) values of the expiratory and/or inspiratory resistance. In some variations the indicator is a color indicator, or the like. In some variations the indicator is a digital signal sent by the device. The indicator does not need to be part of (or coupled to) the control or the resistance modifying member, although it may be part of or coupled to either the control and/or the resistance modifying member. In some variations, the indicator is keyed to the position of the resistance modifying member.

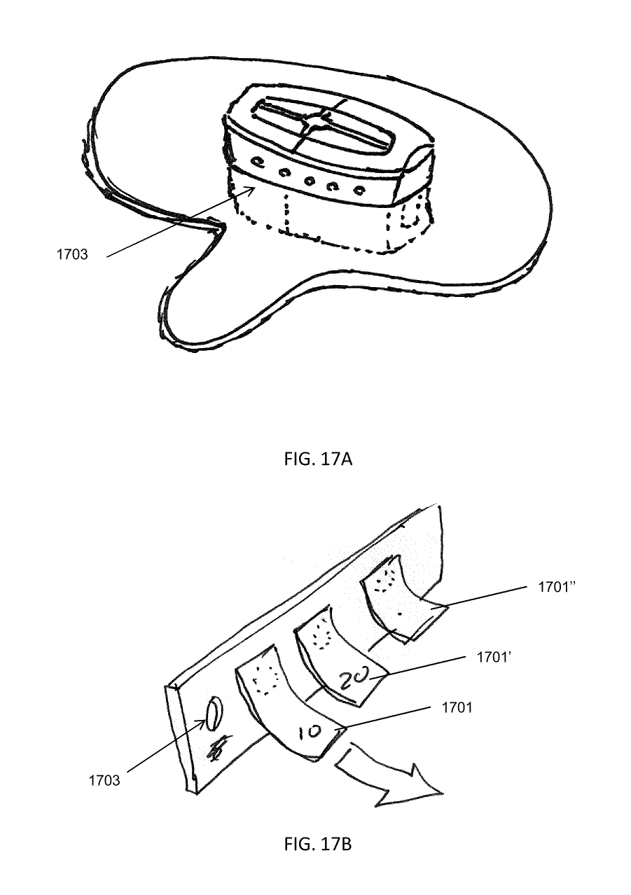

For example, in FIG. 15 the tabs (adhesively removable resistance modifying members 3301 and 3303) may be color coded or may be otherwise labeled (e.g., alphanumerically) with an indicator of the expiratory resistance. Removal of tabs may expose an indictor of the expiratory resistance. FIG. 17B illustrates another variation such as this, in which the pull-tabs (removable expiratory resistance modifying members) are labeled and act as indicators.

FIG. 16B shows another variation of the resistance modifying member shown in FIG. 16A, in which the leak pathway is a single elongate leak pathway 1603′ that is covered more or less (indicated by angle 1605) by turning the dial 1603′. In some variations the resistance modifying member may be configured so that it does not close the leak pathway completely. In both FIGS. 16A and 16B the resistance modifying member does not contact or otherwise interfere with the airflow resistor, and particularly not the moving portions of the airflow resistor.

In FIGS. 16A and 16B, the device may include an indicator such as a labeled region and/or a pointer that indicates the position of the dial. In some variations the window through the dial may reveal an indicator of the expiratory resistance.

FIGS. 17A and 17B illustrate another variation of an adjustable-resistance nasal device in which the resistance modifying member is configured as a removable cover. In this variation, the resistance modifying member is a pull tab 1701 that is configured to cover and be removed to expose a leak pathway or portion of a leak pathway 1703. Multiple pull tabs 1701, 1701, 1701″ may be used, as indicated in FIG. 17B. In general, the different settings of the resistance modifying member may be calibrated, and may also be labeled. For example, in FIG. 17B, sequential pull tabs 1701, 1701′ 1701″ are labeled, which may indicate expiratory resistance and/or flow rate. In some variations (similar to the variation shown in FIG. 15), the tabs overlap, and can therefore be removed in a particular order or to “jump” in decrease of expiratory resistance. Tabs may be removable, and in some variations may be replaceable (reusable) or “new” tabs may be used.

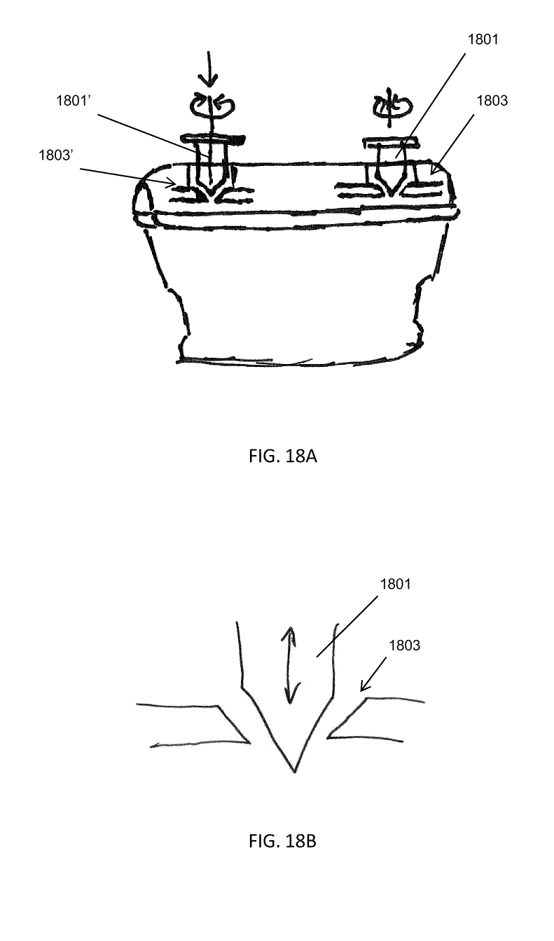

As mentioned above, in some variations the leak pathway is valved to control the expiratory resistance. For example, FIGS. 18A and 18B illustrate one variation of a valved leak path that includes a needle valve 1801 that can be adjusted to increase expiratory resistance (by advancing the pin/needle of the valve into the leak path, as illustrated in FIG. 18B) or to decrease expiratory resistance (by withdrawing the pin/needle of the valve from the leak path). The needle valve may include or be connected to a control such as a knob, dial or the like, to control the movement of the needle in occluding the leak path. For example, the valve may include one or more screws for advancing/withdrawing the needle from the leak path. FIGS. 19A and 19B illustrate another type of valving mechanism that may be used as a resistance modifying member. In this example, a hinged ‘valve’ may be positioned partially over the leak pathway to limit airflow through the device. The hinge may be stiff enough to allow adjustment and maintain the adjusted position. In some variations (e.g. PEEP-type valves), the leak pathway may include a flap-type valve that is weighted to open above a known expiratory pressure.

Adjustable resistance nasal devices such as those described herein may be adapted so that they may be readily adjusted by a third party who is not the subject or patient wearing the device. For example, the adjustable nasal device (or a nasal device that is adjustable by adding or removing a resistance modifying member) may be adjusted by a doctor, nurse or technician (e.g., sleep technician) without disturbing a sleeping subject wearing the device. This may be particularly useful in adjusting a device worn or operated as part of a sleep study. However, this adjustability may also be useful or significant to other third parties (e.g., sleeping partners, spouses, etc.). In addition, the subject himself or herself may also adjust the resistance, which may be helpful in optimizing the comfort or operation of the nasal device.

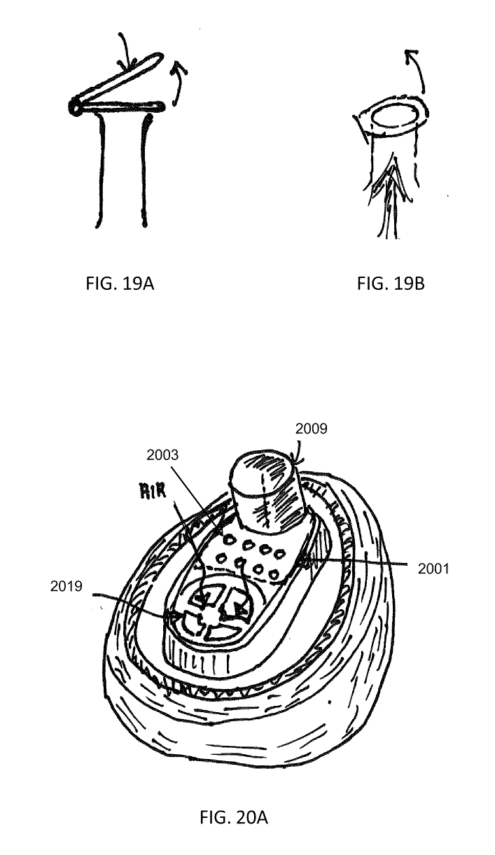

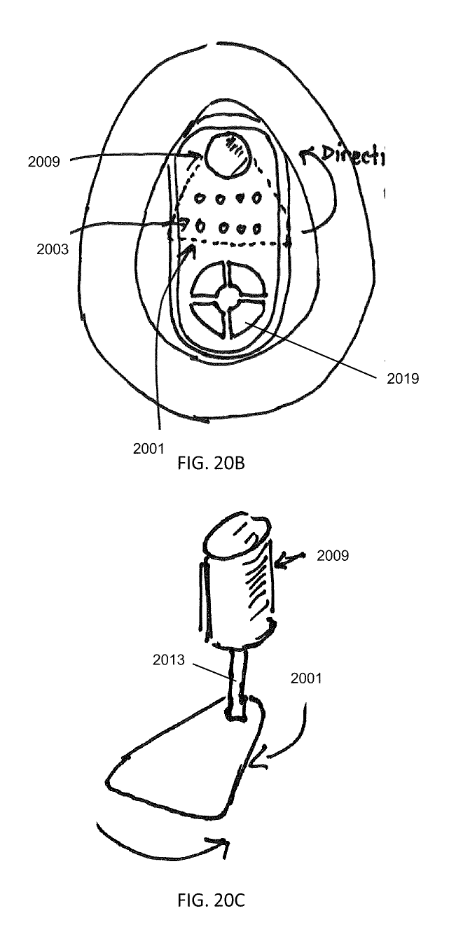

For example, FIG. 20A shows a perspective view of one variation of an adjustable resistance nasal device that is remotely adjustable. This variation includes a driver (servo motor) 2009 that drives a resistance modifying member (rotating cover 2001) that can be rotated to partially or completely occlude one or more leak pathways 2003. The nasal device may include a battery (not shown) to power the motor, and may also include a wireless receiver (not shown) for receiving instructions to drive movement of the resistance modifying member. The airflow resistor 2019 is separate from the adjustable components. In some variations, the device may be controlled by a wired connection extending from the device to a controller. A wireless controller may be used to transmit a control signal to the device, thereby rotating the resistance modifying member to increase or decrease the expiratory resistance by exposing or occluding one or more leak pathways 2003. A top view of this same embodiment is shown in FIG. 20B, and FIG. 20C illustrates one variation of the rotating cover 2001 and driver 2009, shown connected by a drive shaft 2013. Any appropriate driver may be used. For example, in some variation the driver includes an inchworm gear or motor with a piezoelectric actuator that may cover/uncover the cover forming the resistance modifying member. In addition, any appropriate resistance modifying member may be used with a driver. For example, a valved resistance modifying member may also be driven by a driver. In some variation a remotely actuated device may also be locally actuated by actuating a control on the device. In some variations a driver may be used to actuate a resistance modifying member even when it configured only for local activation (e.g., and not remote activation).

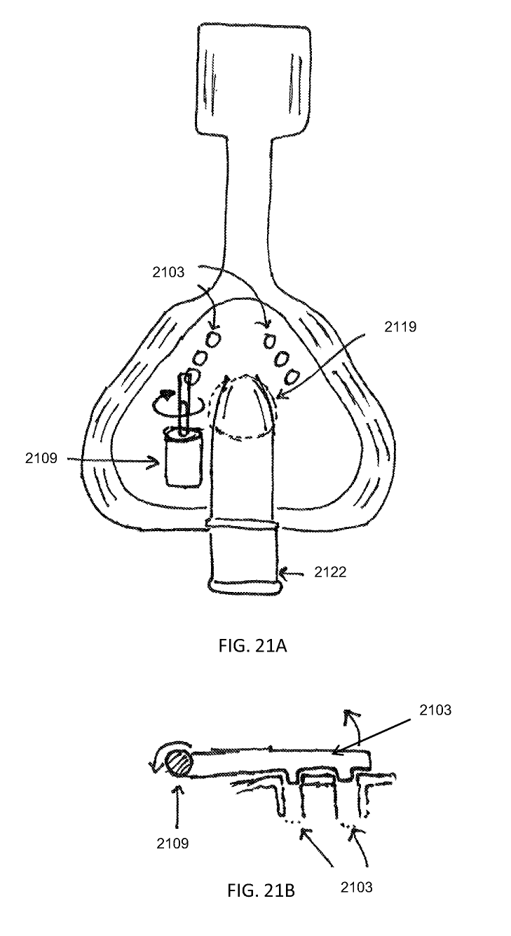

FIGS. 21A and 21B illustrate another variation of an adjustable resistance nasal device configured for remote adjustment of the resistance. In FIG. 21A, the nasal device is configured as a facemask (e.g., a CPAP mask) including a passive airflow resistor 2119 and a plurality of leak pathways 2103. A driver 2109 moves a resistance modifying member, which is a cover 2101 in this example, to expose or occlude the leak pathways 2103. This is illustrated in FIG. 21B. The mask may be connected to a hose 2122 and eventually to a CPAP machine.

### Ramp Kits or Systems

In addition to the adjustable resistance devices described herein, systems or kits including a plurality of nasal devices having fixed expiratory resistances but which increase in resistance relative to each other may also be used. The individual nasal devices may be organized and/or marked in order of increasing expiratory resistance. Such systems or kits may permit a subject to grow accustomed to the increasing expiratory resistance over time by gradually increasing the resistance to exhalation over one or more nights wearing the devices, for some span of time (an acclimation period). The resistance may be increased by any desired amount from a negligible resistance (e.g., a ‘sham’ device) to the final desired expiratory resistance. For example, the resistance of each step may increase by 10% (or 5%, 15%, 20%, 25%, etc.) until the final target expiratory resistance is achieved. This final target expiratory resistance may be approximately 30 cm H₂O/(L/sec), approximately 35 cm H₂O/(L/sec), approximately 40 cm H₂O/(L/sec), approximately 45 cm H₂O/(L/sec), approximately 50 cm H₂O/(L/sec), approximately 55 cm H₂O/(L/sec), approximately 60 cm H₂O/(L/sec), approximately 65 cm H₂O/(L/sec), approximately 70 cm H₂O/(L/sec), approximately 75 cm H₂O/(L/sec), approximately 80 cm H₂O/(L/sec), approximately 85 cm H₂O/(L/sec), approximately 90 cm H₂O/(L/sec), approximately 95 cm H₂O/(L/sec), approximately 100 cm H₂O/(L/sec), approximately 105 cm H₂O/(L/sec), approximately 110 cm H₂O/(L/sec), approximately 115 cm H₂O/(L/sec), although other levels are possible. In one example, the resistance is increased in even steps (e.g., increasing by equivalent amounts between each step), while in some variations the expiratory resistance increases by different amounts between each step, as some increases in expiratory resistance may feel more drastic than others.

Any number of steps of increasing resistance may be used. For example, the number of steps (e.g., the number of different expiratory resistance levels) may depend on the target expiratory resistance, or the period of acclimation. In some variations, two, three, four, five, six, seven, eight, etc. steps may be used. Any number of devices may be used at each step (e.g., any number of devices having the same expiratory resistance) as part of the system or kit. In some variations, each step is ‘held’ for between 1-7 nights. For example, the kit may include three ‘sham’ devices having negligible expiratory resistance, three devices having low expiratory resistance (e.g., 20 cm H₂O/(L/sec)), three devices having a resistance to exhalation that is slightly higher (e.g., approximately 40 cm H₂O/(L/sec)), three devices having a still slightly higher resistance to exhalation (e.g., approximately 60 cm H₂O/(L/sec)), and four devices having an even higher resistance to exhalation (e.g., approximately 80 cm H2O/(L/sec)). In some variations some ‘steps’ may include more than three or less than three devices. In this example, each device is intended to be worn for one night, with devices being worn on consecutive nights. After completing the series of devices, the user may be acclimated to the final resistance and may thereafter use devices having this final (target) resistance.

As mentioned, any of these systems or kits may include instructions for use, indicating that the subject should use the devices in an indicated order which corresponds to an increasing expiratory resistance. The instructions may be included with the devices. In some variations the devices in the kit or system are numbered or otherwise marked to indicate the order to be used. In other variation, the devices are packaged in such a way that they are dispensed or provided in the desired order.

In some variations, there may be excess devices at each step, and the subject may be instructed to remain at a particular step (level of expiratory resistance) until they are comfortable with that level of expiratory resistance, and then proceed to the next higher level. Thus, in any of these variations, the devices corresponding to each step may be labeled sequentially, or marked sequentially via the packaging or dispensing. For example, the devices or set of devices are marked to indicate their order in the sequence (or are packaged to indicate their order in the sequence).

Although the examples of adjustable-resistance nasal devices described above and shown in the figures provided are exemplify the principles taught herein. These same principles may be applied or adapted for use in other nasal device variations. For example, the nasal devices described herein are primarily devices for altering the expiratory resistance of a nasal device. Adjustable nasal devices in which the inspiratory resistance is adjustable (in addition to the expiratory resistance or instead of adjusting the expiratory resistance) are also contemplated as part of this invention. In addition, adjustable-resistance nasal devices may include additional features, which may be combined. For example, an adjustable-resistance nasal device may also include a noise-reducing element and one or more sensor (including a cannula) or the like.

Furthermore, although the nasal devices described herein are configured so that (in normal operation) the resistance through the device is greater during exhalation than during inhalation, other configurations may also be used with the noise-reduced devices or features described herein. For example, a nasal device may be configured with an airflow resistor that inhibits inhalation more than exhalation, which may be used with a noise-reduction element or flap valve configured to inhibit oscillation of the flap (or flaps) during exhalation instead (or in addition to) inhalation. In general a noise-reduced nasal device may limit the oscillation of the flap during both inhalation and exhalation. While the methods and devices have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. An adjustable resistance nasal device comprising:
an airflow resistor configured to inhibit exhalation more than inhalation;
a holdfast configured to secure the nasal device in communication with a subject's nostril;
a leak pathway through the nasal device that is separate from the airflow resistor and is configured to be open during both exhalation and inhalation; and
a control configured to adjust the expiratory resistance through the nasal device.

2. The device of claim 1 further comprising a resistance modifying member configured to modify the leak pathway, wherein the resistance modifying member is controlled by the control.

3. The device of claim 2, wherein the resistance modifying member comprises a shutter configured to at least partially occlude the leak pathway.

4. The device of claim 2, wherein the resistance modifying member comprises a cover configured to expose or cover the leak pathway.

5. The device of claim 4, wherein the cover comprises a pull tab.

6. The device of claim 2, wherein the resistance modifying member comprises an adjustable valve regulating the leak pathway.

7. The device of claim 1, further comprising an indicator configured to indicate the expiratory resistance selected by the control.

8. The device of claim 1, wherein the leak pathway is a constrictable leak pathway configured so that the size leak pathway may be adjusted by the control.

9. The device of claim 1, wherein the control is configured to allow manual adjustment of the expiratory resistance.

10. The device of claim 1, wherein the control is configured to allow remote adjustment of the expiratory resistance.

11. The device of claim 2, further comprising a driver to drive the resistance modifying member.

12. The device of claim 1, further comprising a sensor configured to detect intranasal pressure.

13. An adjustable resistance nasal device comprising:
an airflow resistor configured to inhibit exhalation more than inhalation;
a holdfast configured to secure the nasal device in communication with a subject's nasal cavity;
a leak pathway through the nasal device that is separate from the airflow resistor and configured to be open during both exhalation and inhalation; and
a resistance modifying member configured to allow adjustment of airflow through the leak pathway.

14. The device of claim 13, further comprising an adjustable control configured to adjust the expiratory resistance through the nasal device by controlling the resistance modifying member.

15. The device of claim 13, wherein the adjustable control is a manual control.

16. The device of claim 13, wherein the adjustable control is a remote control.

17. The device of claim 13, removable and replaceable cover configured to occlude the leak pathway.

18. The device of claim 13, wherein the resistance modifying member comprises an adjustable valve.

19. The device of claim 13, wherein the resistance modifying member comprises a removable cover.

20. The device of claim 13, wherein the resistance modifying member comprises a shutter.

21. The device of claim 13, further comprising an indicator configured to indicate the expiratory resistance.

22. An adjustable resistance nasal device comprising:
an airflow resistor configured to inhibit exhalation more than inhalation, wherein the airflow resistor comprises a plurality of flap valves;
an adhesive holdfast configured to secure the nasal device to a subject's nose;
a leak pathway through the nasal device that is separate from the airflow resistor and configured to be open during both exhalation and inhalation; and
a resistance modifying member comprising a removable tab configured to adjust airflow through the leak pathway.

23. The device of claim 22, wherein the airflow resistor and adhesive holdfast form a flexible, substantially planar device.

24. The device of claim 22, further comprising an indicator to indicate the expiratory resistance.

25. An adjustable resistance nasal device system comprising:
a nasal device having an airflow resistor configured to inhibit exhalation more than inhalation, a holdfast configured to secure the nasal device in communication with a subject's nasal cavity, and a leak pathway through the nasal device configured to be open during both exhalation and inhalation; and
a resistance-modifying member configured to secure to the nasal device and at least partially occlude the leak pathway.

26. The system of claim 25, wherein the resistance modifying member is a snap-on resistance modifying member configured to mechanically secure to the nasal device.

27. The system of claim 25, wherein the resistance modifying member is an adhesive resistance modifying member configured to adhesively secure to the nasal device.

28. An adjustable resistance nasal device comprising:
an airflow resistor configured to inhibit exhalation more than inhalation;
a holdfast configured to secure the nasal device in communication with a subject's nasal cavity; and
a constrictable leak pathway through the nasal device configured to be open during both exhalation and inhalation.

29. The device of claim 28, wherein the constrictable leak pathway comprises an inflatable bladder.

30. The device of claim 28, wherein the constrictable leak pathway comprises a swellable material.

31. A method of controllably adjusting the resistance of a nasal device, wherein the nasal device comprises an airflow resistor configured to have a greater resistance to exhalation than inhalation, a holdfast for securing the nasal device in communication with a subject's nasal cavity and a leak pathway separate from the airflow resistor that is configured to be open during both exhalation and inhalation, the method comprising adjusting the airflow through the leak pathway.

32. The method of claim 31, further comprising providing an indicator of the resistance to exhalation.

33. The method of claim 31, wherein the step of adjusting the airflow through the leak pathway comprises at least partially occluding or opening the leak pathway.

34. The method of claim 31, further comprising adjusting a control on the nasal device to adjust the airflow through the leak pathway.

35. The method of claim 31, further comprising adjusting a control that is remote from the device to adjust the airflow through the leak pathway.

36. The method of claim 31, further comprising driving an actuator to adjust the airflow through the leak pathway.

37. The method of claim 31, further comprising manipulating a resistance modifying member to adjust airflow through the leak pathway.

38. The method of claim 37, wherein the resistance modifying member comprises a cover that may be removed or applied to the leak pathway to adjust airflow through the leak pathway.

39. The method of claim 37, wherein the resistance modifying member comprises a shutter configured to at least partially occlude the leak pathway.

40. The method of claim 37, wherein the resistance modifying member comprises an adjustable valve regulating the leak pathway.

41. The method of claim 31, wherein the step of adjusting the airflow through the leak pathway comprises opening or closing the leak pathway.

42. The method of claim 31, wherein the step of adjusting the airflow through the leak pathway comprises constricting or dialing the leak pathway.

43. A method of controllably adjusting the resistance of a nasal device, wherein the nasal device comprises an airflow resistor configured to have a greater resistance to exhalation than inhalation, a holdfast for securing the nasal device in communication with a subject's nasal cavity and a leak pathway separate from the airflow resistor that is configured to be open during both exhalation and inhalation, the method comprising:
adjusting the airflow through the leak pathway; and
indicating the resistance to exhalation.

44. The method of claim 43, further comprising adjusting the airflow through the leak pathway in response to the resistance to exhalation.
